(19) United States
(12) Patent Application Publication
(10) Pub. No.: US 2021/0215631 A1
(43) Pub. Date: Jul. 15, 2021

(54) BUBBLE-BASED ELECTROCHEMICAL METHODS FOR THE ENRICHMENT AND DETECTION OF SURFACTANTS IN AQUEOUS SOLUTIONS

(71) Applicant: WAYNE STATE UNIVERSITY, Detroit, MI (US)

(72) Inventors: Long Luo, Troy, MI (US); Ruchiranga Ranaweera, Detroit, MI (US)

(73) Assignee: WAYNE STATE UNIVERSITY, Detroit, MI (US)

(21) Appl. No.: 17/148,392

(22) Filed: Jan. 13, 2021

Related U.S. Application Data
(60) Provisional application No. 62/960,523, filed on Jan. 13, 2020.

(51) Int. Cl. G01N 27/30; G01N 33/18; G01N 1/38

(52) U.S. Cl. CPC G01N 27/30; G01N 33/1826; B82Y 30/00; G01N 1/38; G01N 33/182

(57) ABSTRACT

Bubble-based systems and methods to detect surfactants, such as perfluorooctanesulfonate (PFOS) and perfluorooctanoic acid (PFOA) in aqueous solutions including water supplies are described. The systems and methods can utilize a surfactant preconcentration step to increase the sensitivity of the methods. Preconcentration can be based on aerosol formation and capture.

### CROSS REFERENCE TO RELATED APPLICATION

This application claims priority to U.S. Provisional Patent Application No. 62/960,523 filed on Jan. 13, 2020, which is incorporated herein by reference in its entirety as if fully set forth herein.

### FIELD OF THE DISCLOSURE

The current disclosure provides bubble-based systems and methods to detect surfactants, such as perfluorooctanesulfonate (PFOS) and perfluorooctanoic acid (PFOA) in aqueous solutions, including water supplies. The systems and methods can utilize a surfactant preconcentration step to increase the sensitivity of the methods. Preconcentration can be based on aerosol formation and capture.

### BACKGROUND OF THE DISCLOSURE

Access to safe drinking water is a major concern for both developed and developing countries due to the link between contaminated drinking water and the transmission of disease. Environmental pollutants, such as toxic organic chemicals, have the potential to threaten the safety and sanitation of drinking-water supplies. Specifically, exposure to organic chemicals, such as perfluorooctanesulfonate (PFOS) and perfluorooctanoic acid (PFOA), in contaminated drinking-water, can lead to adverse human health effects. These health effects include: low infant birth weights; adverse effects on the immune system; cancer; and thyroid hormone disruption.

Per and poly-fluorinated surfactants are synthetic organofluorine chemical compounds that have been used in many industries worldwide, such as in the making of stain and grease repellents and firefighting foams. The use and emission of these surfactants by manufacturing industries have contributed to PFOS and PFOA as the dominant contaminants in drinking water. Although such surfactants are no longer manufactured in the United States, the strong carbon-fluorine bonds which make up their chemical structure contribute to a long environmental half-life and continued bioaccumulation in humans and the environment. When interacting with the human body, PFOS and PFOA are well absorbed orally but poorly eliminated. The US Environmental Protection Agency (EPA) reported a laboratory study that showed the PFOS half-life in the human body is 5.4 years.

Considering these findings, the EPA developed risk management strategies to control the reintroduction of poly-fluorinated surfactants into the US. Among the risk management strategies, the EPA established a health advisory for PFOS and PFOA to provide interested stakeholders with information on these surfactants' health risks. In addition, the EPA established methods for detecting PFOS and PFOA in drinking water. Specifically, the EPA Unregulated Contaminant Monitoring Rule (UCMR 3) required that “Method 537” be used to detect and analyze PFOS and PFOA in drinking water. Method 537 utilizes solid-phase extraction to preconcentrate the surfactant in solution and liquid chromatography/tandem mass (LC/MS/MS) spectrometry to detect PFOS and PFOA in solution. The health advisory levels reported by the EPA for PFOS and PFOA in drinking-water, individually or combined, was 70 ng/L.

A wide range of additional methodologies have been employed to detect and quantify PFOS and PFOA in drinking water, in addition to the US EPA's Method 537. Other methodologies include the use of liquid and gas chromatography. For example, CN103487515A describes a method of immunoaffinity chromatography-ultrahigh performance chromatography-mass spectrum to detect perfluorinated compounds in dairy products or water bodies.

WO2019169177A1 describes a method of using ultra-performance liquid mass spectrometer tandem quadrupole mass spectrometry (UPLC-MS/MS) to detect PFOS and PFOA in aqueous (such as groundwater) and non-aqueous (such as soil) environmental samples. CN103487584A describes utilizing enzyme-linked immunosorbent assay (ELISA) to detect perfluorooctane sulfonate residual in environmental samples, such as water.

The current use of LC/MS/MS, liquid and gas chromatographs, and ELISAs have drawbacks, however, for surfactant determination in aqueous environmental samples. Method 537 and liquid and gas chromatography, for example, require the use of expensive and complicated instruments and extensive sample preparation steps. They can also rely on large amounts of solvents during analysis. ELISAs provide high detection limits compared to other methodologies used to detect surfactants in environmental samples, such as water. As a result, there remains a need for improved chromatography methods to detect and quantify surfactants in aqueous solutions.

### SUMMARY OF THE DISCLOSURE

The present disclosure provides at least two new advances for detecting and quantifying surfactants in aqueous solutions. One provides for the use of bubble nucleation-based electrochemical methods to detect and quantify surfactants in aqueous solutions. The second increases the sensitivity of surfactant detection methods by providing a novel aerosol-based surfactant preconcentration step. These two advances can be individually combined with other previously available techniques. For example, the bubble nucleation-based electrochemical methods to detect and quantify surfactants can be used with a different preconcentration step, such as solid-phase extraction. Similarly, the aerosol-based surfactant preconcentration step can be used with a different detection and quantification step, such as mass spectrometry. Regardless of the combination chosen, the advances provided by the current disclosure simplify and streamline currently available methods to detect and quantify surfactants within aqueous solutions.

Bubble-nucleation refers to bubble formation within a liquid. According to classical nucleation theory (CNT), the formation of a gas bubble requires a supersaturation of dissolved gas within a liquid to overcome the high energy barrier of establishing a new gas-liquid interface. However, surfactants lower the surface tension of aqueous solutions such as water to reduce the energy barrier to establishing new gas-liquid interfaces. Accordingly, bubbles can form in liquids with surfactants at a lower gas concentration than if they were not present. The current disclosure takes advantage of this relationship to detect surfactants' presence and concentration within an aqueous solution.

In practice, the method includes placing an electrode within an aqueous solution, supplying a voltage to the electrode, and monitoring current values flowing through the electrode based on the applied voltage. The voltage application produces a gas evolution reaction by separating chemical species within the aqueous solution to generate gas bubbles. While the voltage is being applied, the monitored current will increase until it reaches the peak current value or iₚₑₐₖ. Past the peak current value, the current quickly drops to a minimal value. This drop to a minimal value indicates bubble formation at the electrode's surface that impedes continued current flow.

Monitored current values, such as the peak current value, the minimal current value following the peak, and the difference between these two values are proportional to the gas saturation level required for bubble formation. That is, as surfactant concentrations increase within an aqueous solution, the gas saturation level decreases. This leads to an inverse relationship where a lower peak current value correlates with a higher surfactant concentration in an aqueous solution. Similarly, the minimal peak current value is inversely correlated to the surfactant concentration in the aqueous solution. A smaller difference between the peak current value and the minimal current value also correlates with a higher surfactant concentration in the aqueous solution.

The current disclosure takes advantage of one or more of these relationships by transducing the change in gas saturation level required for bubble formation based on the presence of a surfactant to an electrochemical signal that allows determination of the surfactant concentration in the solution. These methods provide a low-cost, sensitive, and even portable means of in situ analysis of surfactants in systems such as waterways.

The current disclosure also provides a novel aerosol-based surfactant preconcentration step. This aspect of the disclosure takes advantage of the fact that surfactants accumulate in a solution at air-solution interfaces. When an electric potential is applied to one or more electrodes to generate gas bubbles, gas bubbles accumulate in the aqueous solution. Surfactants, such as PFOS and PFOA, absorb and accumulate on the surface of the gas bubbles. A thin layer of liquid surrounding the bubble is ejected into the air when the bubbles burst at the aqueous solution's surface. This aerosol droplet contains a high PFOS and PFOA concentration and can be captured and analyzed, for example, using mass spectrometry.

These advances simplify and streamline the processes available to detect and quantify the presence of surfactants in aqueous solutions. They can be used to detect surfactants, such as PFOS and PFOA, at an EPA acceptable level (and beyond) for surfactants in drinking water.

### BRIEF DESCRIPTION OF THE SEVERAL VIEWS OF THE DRAWINGS

Many of the drawings submitted herein are better understood in color. Applicant considers the color versions of the drawings as part of the original submission and reserves the right to present color images of the drawings in later proceedings.

FIG. 1: Bubble-based electrochemical method for surfactant detection. The high surface activity of surfactant molecules stabilizes H₂ bubble nuclei, leading to a reduced nucleation barrier.

FIG. 2: Preconcentration of PFAS via electrochemical aerosol formation. The high surface activity of PFAS allows PFAS to spontaneously adsorb and accumulate on the surface of gas bubbles. When the bubbles burst, only a thin layer of liquid surrounding the bubbles is ejected into the air resulting in a high concentration of PFAS in the aerosol droplets.

FIGS. 3A; 3B: (3A) The cyclic voltammograms for an 11 nm radius Pt nanoelectrode in 1.0 M HClO₄ containing 0.1 M NaClO₄ and various PFOS concentrations (g/L) including: 0, 10⁻⁴, 5×10⁻⁴, 10⁻³, 5×10⁻³, 10⁻², 5×10⁻², and 10⁻¹. The scan rate is equal to 100 mV/s. (3B) The plot of iₚₑₐₖ vs. C_{PFOS}. Error bars are the standard deviations at each C_{PFOS} from at least three measurements. The best fit of the data points is plotted with R²=0.92, which has a slope of −0.82 nA/dec. The horizontal line shows the mean value of iₚₑₐₖ in the absence of PFOS. The horizontal line's corresponding standard deviation is the thick horizontal line in which the horizontal line is encompassed. The Limit of Detection (LOD) based on 3 times the standard deviation of the blank is calculated to be 80 μg/L.

FIG. 4: Plot of peak vs. C_{PFOA} obtained using a 25-nm-radius Pt nanoelectrode. Error bars are the standard deviations at each C_{PFOA} from at least three measurements. The tilted line is the best fit of the data points with R²=0.9 and a slope of −3.2 nA/dec. The horizontal solid line shows the mean value of iₚₑₐₖ in the absence of PFOA. The solid line's corresponding standard deviation is the thick horizontal line in which the solid line is encompassed. The LOD based on three times the standard deviation of the blank is calculated to be 30 μg/L.

FIG. 5: Plots of the normalized peak current (iₚₑₐₖ/i⁰ₚₑₐₖ) vs. the concentration of perfluorinated carboxylic acids (C_{PFCA}) with different alkyl chain lengths. i⁰ₚₑₐₖ is the peak current at C_{PFCA}=0.

FIGS. 6A-6D. iₚₑₐₖ vs. surfactant concentration for different perfluoroalkylcarboxylic acids (PFCA). Only PFOA and PFHpA showed a linear relationship. Nanoelectrode radii used in these experiments are PFOA (25 nm), PFHpA (20 nm), PFHxA (14 nm), and PFBA (16 nm). The solid horizontal lines and the shaded regions indicate the mean values of iₚₑₐₖ and their corresponding standard deviations in the absence of PFCA.

FIG. 7: Photographs show pendant drop shapes of the 1.0 M HClO₄/0.10 M NaClO₄ solution with 0.10 g/L perfluorinated carboxylic acids with different alkyl chain lengths and the corresponding surface tension data.

FIGS. 8A-8D: iₚₑₐₖ vs. surfactant concentration for different perfluoroalkyl sulfonates (PFAS). Only PFOS and PFHpS showed a linear relationship. Nanoelectrode radii used in these experiments are PFOS (11 nm), PFHpS (18 nm), PFHxS (15 nm), and PFBS (10 nm). The solid horizontal lines and the shaded regions indicate the mean values of iₚₑₐₖ and their corresponding standard deviations in the absence of PFAS.

FIG. 9: Photographs show pendant drop shapes of 1.0 M HClO₄/0.10 M NaClO₄ solution with C_{PFOS} from 10⁻⁶ to 10 g/L.

FIGS. 10A, 10B: (10A) Surface tension of PFOS-containing HClO₄ solutions measured by the pendant drop method. The best fit of the data points for C_{PFOS}=10⁻⁴ to 10 g/L is represented by the solid black line with R²=0.99 and a slope of −9.8 mN/m·dec. (10B) Comparison of experimental data and theoretical fit in the form of Eq 6.

FIG. 11: Plot of iₚₑₐₖ vs. C_{PFOS} for PFOS samples before and after preconcentration using solid-phase extraction (SPE). The data after SPE is linearly fitted with R²=0.92 and a slope of −1.1 nA/dec. The horizontal solid line shows the mean value of iₚₑₐₖ in the absence of PFOS. The corresponding standard deviation of the solid line is the thick horizontal line in which the solid line is encompassed. The LOD based on 3 times the standard deviation of the blank is calculated to be 40 ng/L.

FIG. 12: Cyclic voltammograms for a 10-nm-radius Pt nanoelectrode in 1.0 M HClO₄ containing 0.1 M NaClO₄ and various PFOS concentrations (g/L): 0, 10⁻⁷, 10⁻⁴, and 10⁻⁷ after SPE preconcentration. Scan rate=100 mV/s.

FIG. 13A, 13B: (13A) Cyclic voltammograms and (13B) the corresponding average iₚₑₐₖ for a 7 nm radius Pt nanoelectrode in 1.0 M HClO₄ containing 0.1 M NaClO₄, 1.0 mg/L PFOS, and a 10- to 1000-fold excess of poly(ethylene glycol) (PEG, 400 g/mol). The scan rate=100 mV/s.

FIGS. 14A-14C: Plots of the normalized peak current (iₚₑₐₖ/i⁰ₚₑₐₖ) vs. the concentration of (14A) humic acid, (14B) lysozyme, and (14C) Tween-20. i⁰ₚₑₐₖ is the peak current of the blank. Horizontal bands show the standard deviation of the blank.

FIGS. 15A-15D: Photographs of the experimental setup for electrochemical aerosol enrichment. (15A) A home-built two-compartment electrochemical cell with a total volume of 700 mL, (15B) H₂ microbubbles generated at a 1.5 cm×1.5 cm Ni foam electrode immersed at a depth of 25 cm and a voltage of 70 V, (15C) a 3 mm separation between the water surface and the aerosol collection glass slide, and (15D) the collected aerosol droplets on the glass slide after 5 min.

FIG. 16: Plots of r_{bubble} vs. h at various voltages from 30 V to 80 V. The error bars represent the radii's standard deviations of at least 50 randomly selected gas bubbles. The average r_{bubble} for each voltage was calculated from the mean values of r_{bubble} at all bubble path lengths (h).

FIGS. 17A, 17B: (17A) Mass spectra of diluted aerosol sample, two PFOS standards (5×10⁻⁷ M and 10⁻⁸ M in a 50/50 v/v H₂O and MeOH mixture), and the blank in the negative ion detection mode. The aerosol sample was collected at 70 V for 10 min using 10⁻⁸ M PFOS solution as the electrolyte solution. The signals at m/z=498.8 correspond to [PFOS−H]⁻. (17B) Enrichment factor, R, as a function of the PFOS concentration in the bulk solution (C_{PFOS, bulk}). R is defined as the ratio of PFOS concentrations in the aerosol (C_{PFOS, aerosol}) and its corresponding C_{PFOS, bulk}. The thick horizontal line in which the horizontal solid line is encompassed shows the average and standard deviation of Rs at all concentrations. The error bars are the standard deviations of three independent injections of the collected aerosol samples at each concentration.

FIG. 18: Electrospray ionization mass spectra of standard solutions of PFHxA, PFHpA, PFOA, PFNA, PFDA, PFUnDA, PFDoDA, PFBS, and PFHxS. Each sample has a concentration of 10⁻⁵ M in a 50/50 v/v H₂O and MeOH mixture.

FIG. 19: Calibration curves for 3 different perfluoroalkyl sulfonic acids.

FIG. 20: Enrichment factor, R, for eight independent enrichment experiments using a 10⁻¹⁰ M PFOS bulk solution. Experimental conditions were identical to those in FIGS. 17A, and 17B. Statistics of R values: mean=1110, standard deviation=90, and between-run coefficient of variation=8%.

FIG. 21: Calibration curves for 7 different perfluoroalkyl carboxylic acids.

FIG. 22: The mass-to-charge (m/z) signals used for quantifying 10 common PFAS.

FIG. 23: Factor, R, for 10 common PFAS including 7 perfluorinated carboxylic acids with carbon chain lengths from 6 to 12 (PFHxA, PFHpA, PFOA, PFNA, PFDA, PFUnDA, and PFDoDA) and 3 perfluorinated sulfonic acids (PFBS, PFHxS, and PFOS) at C_{PFAS, bulk}=10⁻¹⁰ M. Experimental conditions were identical to those in FIGS. 17A, and 17B.

FIG. 24: Enrichment factor, R, for 9 common PFAS at different bulk concentrations. The shaded boxes show the average and standard deviation of Rs at all concentrations.

FIG. 25: Plots of R vs. h at different C_{PFOS, bulk} from 10⁻¹¹ to 10⁻⁸ M. At C_{PFOS, bulk}=10⁻¹¹, 10⁻¹⁰, and 10⁻⁹ M, there is very similar linearity between R and h with a slope of 30 cm⁻¹ and a y-intercept of 250. At C_{PFOS, bulk}=10⁻⁸ M and h>20 cm, R start deviating from such linearity.

FIGS. 26A, 26B: (26A) The “total adsorption” model proposed by Chen, et al. (Chingin, ACS Omega 2018, 3, 8709-8717). This model assumes that the liquid near a bubble was constantly renewed due to bubble movement, and every surfactant that meets a rising bubble was adsorbed. (2B) Diffusion-limited adsorption model. The model assumes the liquid near a bubble is not renewed during the rise of a bubble. The bubble surface acts as a PFAS sink. Diffusion of PFAS limits the adsorption rate.

FIGS. 27A, 27B: (27A) Simulated C_{PFOS}/C_{PFOS, bulk} near a 70-μm-radius gas bubble as a function oft. (27B) The amount of PFOS adsorbed on the surface of a bubble (n_{PFOS}) as a function of t when C_{PFOS, bulk}=10⁻¹¹ M.

FIG. 28: Enrichment rate, ∂R/∂h, as a function of r_{bubble} and the fit of experimental data in the form of Eq 6. The average radii of bubbles, r_{bubble}, was tuned by applying different voltages from 30 to 80 V. Other experimental conditions: h=25 cm and C_{PFOS, bulk}=10⁻¹⁰ M.

FIG. 29: Mass spectrum of tap water. No PFAS signal was observed. The background signals at m/z=255.1 and 283.2 belong to the palmitate and stearate ions.

FIGS. 30A, 30B: (30A) Mass spectra of two 20-fold diluted aerosol samples, which were collected at 70 V for 10 min using PFAS-spiked tap water (top) and clean tap water without spiking (bottom) as the electrolyte solution, respectively. The signals at m/z=368.9, 398.9, and 498.8 correspond to [PFOA-CO2-H]⁻, [PFHxS-H]⁻, and [PFOS-H]⁻, respectively. The tap water was collected at the chemistry building of Wayne State University. After spiking, the tap water contained a mixture of 0.04 nM (16 ng/L) PFHxS, 0.02 nM (8 ng/L) PFOA, and 0.2 nM (100 ng/L) PFOS. (30B) PFAS concentrations before and after preconcentration. The measured preconcentration factor, R_{measured}, vs. the expected preconcentration factor, R_{expected}, for each PFAS compound spiked in the tap water. The R_{expected} values are the average R values for each PFAS compound at concentrations from 10⁻¹² to 10⁻⁹ M.

FIG. 31: Geometry of mesh used in the finite element simulation.

### DETAILED DESCRIPTION

Per- and polyfluorinated alkyl substances (PFAS) are a group of anthropogenic compounds, some of which have been manufactured since the 1950s (Mejia-Avendaño, et al., Anal. Chem. 2017, 89, 2539-2546). Due to their unique oleophobic and hydrophobic properties and high stability, PFAS are widely used in textile, upholstery, nonstick product manufacturing, aqueous film forming foams, and hydraulic fluids (Seow, J., Hemming Information Services, 2013; Fiedler, et al., Toxicol. Environ. Chem. 2010, 92, 1801-1811). Widespread use and extreme resistance to degradation have resulted in these compounds' ubiquitous presence in the environment. According to previous studies (Wang, et al., Environ. Int. 2014, 70, 62-75; Paul, et al., Environ. Sci. Technol. 2009, 43, 386-392), the cumulative global emissions of PFAS are at least 46,000 tons with a large fraction released directly to water systems. Recent results have shown PFAS can activate oxidative stress, which is related to several diseases, or events in humans, including atherosclerosis, heart attack, chronic inflammatory diseases, central nervous system disorders, age-related disorders, and cancer (Berthiaume, et al., Toxicol. Lett. 2002, 129, 23-32; Hu, et al., Toxicol. Sci. 2002, 68, 429-436; Wielsøe, et al., Chemosphere 2015, 129, 239-245; Richardson, et al., Anal. Chem. 2018, 90, 398-428). Because of PFAS-related health concerns, the U.S. Environmental Protection Agency (EPA) published a health advisory for PFOS and PFOA—the two most common PFAS—in drinking water to be 70 ng/L individually or combined in 2016. Following the EPA, some state governments have published stricter health advisories than the EPA standard. For example, Vermont's health advisory level for the sum of five PFAS was set not to exceed 20 ng/L in drinking water. More recently, the state congress of Pennsylvania proposed that the State Environmental Quality Board adopt a maximum PFAS contaminant level as low as 5 ng/L.

Detection of 5 ng/L PFAS in water poses an analytical challenge. The most commonly used analytical method for PFAS is EPA Method 537. This method was established to determine 18 different PFAS in drinking water using solid-phase extraction and liquid chromatography/tandem mass spectrometry. This method's reported detection limit varies among different laboratories but is typically a few ng/L, which is just around the desired detection limit of 5 ng/L (Prakash, et al.; “Comparison of ASTM by the ASTM Method versus Solid Phase Extraction”; Eurofins Eaton Analytical, 2018). These detection limits are achieved by a 250-fold preconcentration using a multistep and time-consuming (up to several hours) solid-phase extraction before mass spectrometry detection. Therefore, there is a critical need for a rapid and efficient preconcentration method for PFAS analysis. The current disclosure describes two new ways to determine the concentration of surfactants (such as PFAS) in an aqueous solution. The first method involves the use of a bubble-based electrochemical method for rapid surfactant detection. The second method involves an efficient preconcentration method for surfactant detection, based on electrochemical aerosol formation.

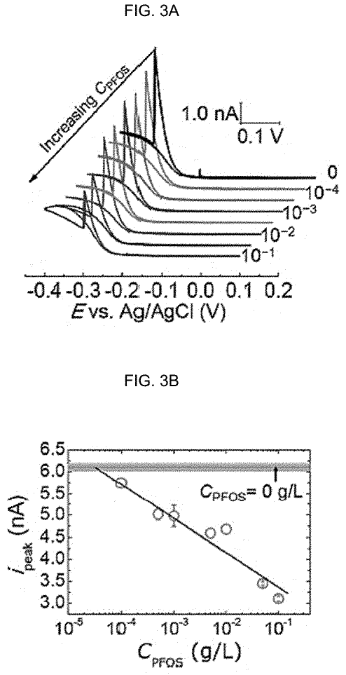

In the first method, bubble-nucleation is used to determine the concentration of surfactants in the solution. Bubble-nucleation is a thermodynamic process that involves bubble formation in electrochemical systems. Electric potential is applied to an electrode in an aqueous solution, which produces a gas evolution reaction by separating chemical species within the aqueous solution to generate gas bubbles such as hydrogen gas, oxygen gas, or nitrogen gas bubbles. With the continued application of voltage, the current increases until it precipitously drop (see, for example, FIG. 3A). The drop in current indicates nucleation and formation of gas bubbles at the electrode surface, thereby blocking the electrode's surface and the associated current. Monitored current values, such as the current drop, minimal current value, and peak current value (also referred to herein as iₚₑₐₖ), are inversely correlated with the presence and amount of surfactant within the solution. For example, a higher iₚₑₐₖ value inversely correlates with less surfactant within the aqueous solution.

In the second method, surfactants are preconcentrated on bubbles formed by water electrolysis. The bubbles burst at the air-solution interface forming an aerosol enriched with surfactants. This method is based on the natural phenomenon of sea-spray aerosol enrichment (Aller, et al., Aerosol Sci. 2005, 36, 801-812; McMurdo, et al., Environ. Sci. Technol. 2008, 42, 3969-3974; Bertram, et al., Chem. Soc. Rev. 2018, 47, 2374-2400).

In sea-spray aerosol enrichment, the ocean wind causes a near-surface velocity gradient in the water column that results in wave breaking. The entrainment of air into the water column produces a plume of bubbles. These bubbles serve to scavenge surface-active material, carrying it to the air-ocean interface, where the bubbles burst and form a sea-spray aerosol (Wilson, et al., Nature 2015, 525, 234). These aerosol particles are enriched in surface-active organic materials such as free fatty acids and anionic surfactants (McMurdo, et al., Environ. Sci. Technol. 2008, 42, 3969-3974; Bertram, et al., Chem. Soc. Rev. 2018, 47, 2374-2400). Recently, Chen and co-workers (Chingin, et al., Anal. Chem. 2016, 88, 5033-5036; Chingin, et al., Metabolomics 2016, 12, 171; Chingin, et al., ACS Omega 2018, 3, 8709-8717) mimicked this phenomenon by generating gas bubbles in water using an air diffuser and collected the aerosol droplets formed by bubble bursting. They found the organic solutes in the aerosol were enriched by 6 to 12-fold for organic metabolites in urine (e.g., lipids and lipid-like molecules, phenylpropanoids and polyketides) (Chingin, et al., Metabolomics 2016, 12, 171), 20 to 1000-fold for rhodamine dyes (Chingin, et al., ACS Omega 2018, 3, 8709-8717), and 10 to 100-fold for amino acids, protein, and DNA (Chingin, et al., Anal. Chem. 2016, 88, 5033-5036).

Inspired by these previous studies, a preconcentration method for surfactants based on electrochemical aerosol formation was developed. Rather than using an external gas supply to generate bubbles, H₂ bubbles were in situ electrogenerated by water reduction. The advantages of the design are 3-fold. First, electrogeneration of gas bubbles simplifies the design, making miniaturization easy. Second, it allows additional modulation of the bubble formation by electrochemistry (e.g., controlling the bubble sizes and affecting the surfactant and electrode interactions by electrode potential). Third, it also improves the reproducibility of the aerosol-based enrichment by minimizing the initial momentum of gas bubbles, which helps to achieve a predictable low Reynolds number motion of bubbles (Magnaudet, et al., Annu. Rev. Fluid Mech. 2000, 32, 659-708; Taqieddin, et al., J. Electrochem. Soc. 2017, 164, E448-E459; Taqieddin, et al., J. Electrochem. Soc. 2018, 165, E694-E711), and by reducing random bubble coalescence, which often occurs when using a porous frit to generate bubbles (Wang, et al., Proc. Natl. Acad. Sci. U.S.A 2017, 114, 6978-6983). In this study, 1000-fold preconcentration of PFAS was demonstrated within 10 min using the method, taking advantage of the high surface activities of PFAS (Shinoda, et al., J. Phys. Chem. 1972, 76, 909-914).

Various components and parameters are involved in carrying out surfactant detection via bubble-nucleation and enrichment of a surfactant in an aerosol. Such components and parameters can include: the surfactant for detection; the aqueous solution housing the surfactant; an electrode; the applied electric potential (e.g., voltage); the formation of gas bubbles; the formation and capture of an aerosol from the gas bubbles; and the analysis of the enriched aerosol via a detection method, such as mass spectrometry. Aspects of these components and parameters are now described with additional detail and options as follows: (i) Surfactants for Detection and Aqueous Solutions; (ii) Bubble Nucleation Conditions; (iii) Preconcentration based on Aerosol Formation and Capture; (iv) Mass Spectrometry Analysis of Preconcentrated Samples; (v) Experimental Examples; and (vi) Closing Paragraphs.

(i) Surfactants for Detection and Aqueous Solutions. Systems and methods can be used to detect the presence of any surfactants within an aqueous solution so long as the surfactant reduces the surface tension of the aqueous solution, thereby reducing the nucleation condition. Surfactants generally lower the surface tension of an aqueous solution due to having a hydrophilic outer layer (head) and a hydrophobic inner layer (tail).

Surfactants detected by systems and methods of the current disclosure can have a carbon chain length of at least n=3. In particular embodiments, the carbon chain length of surfactants includes at least n=3, 5, 6, or 7. Additional carbon chain lengths of detected surfactants can range between 2 to 80. In certain embodiments, detected surfactants are saturated or unsaturated long-chain fatty acids or acid salts, long-chain alcohols, polyalcohols, linear or branched carboxylic acids and acid salts having from 4 to 50 carbon atoms, linear and branched alkyl sulfonic acids and acid salts having from 4 to 50 carbon atoms, linear alkyl benzene sulfonate wherein the linear alkyl chain includes from 4 to 50 carbon atoms, sulfosuccinates, phosphates, phosphonates, phospholipids, ethoxylated compounds, carboxylates, sulfonates and sulfates, polyglycol ethers, amines, salts of acrylic acid, pyrophosphate, and mixtures thereof.

Particular embodiments include detecting ionic surfactants, anionic surfactants, zwitterionic surfactants, nonionic surfactants, alkylbenzenesulfonates surfactants, stearyl sulfate surfactants, sodium lauroyl sarcosinate surfactants, and/or mixtures thereof within an aqueous solution. In particular embodiments, surfactants detected in an aqueous solution include perfluorooctanesulfonate and/or perfluorooctanoate. In particular embodiments, surfactants detected in an aqueous solution include perfluorohexanoic acid, perfluoroheptanoic acid, perfluorooctanoic acid, perfluorononanoic acid, perfluorodecanoic acid, perfluoroundecanoic acid, perfluorododecanoic acid, perfluorobutane sulfonic acid, perfluorohexane sulfonic acid, perfluorooctane sulfonic acid, perfluoroalkyl substance and/or polyfluoroalkyl substance.

Additional surfactants that can be detected include a pH-dependent carboxylate anion, sulfate, sulfonate, hexadecyl sultanate, phosphate, alkyl sulfate, lauryl sulfate, sodium lauryl sulfate, sodium laureth sulfate, sodium myreth sulfate, docusate, tetradecyl sodium lauryl sulfate, 7-ethyl-2-methyl-undecyl sulfate, sulfonate fluorine-containing surfactant, alkyl ether sulfate, alkylaryl ether phosphate, alkyl ether phosphate, alkyl carboxylate, dimethylpolysiloxane, polymethylhydrosiloxane, sodium stearate, octadecanoic acid, sodium oleate, fatty acid salt, fluorine-containing surfactant, and/or mixtures thereof. Such surfactants are known in the art and are described in, for example, CA11886586A and CN105102629A.

The aqueous solution housing the surfactant can be a solvent. In particular embodiments, the aqueous solution includes the sample to be analyzed for the presence of the surfactant. In particular embodiments, the solvent is water. The water can be tap water, river water, seawater, lake water, glacier water, ocean water, salt water, natural water, sound water, strait water, channel water, gulf water, estuary water, polynya water, bay water, inlet water, shoal water, ice water, and/or rainwater. Additionally, the aqueous solution can be acidic or basic.

In particular embodiments, the aqueous solution includes an electrolyte, such as a mixture of sodium perchlorate and perchloric acid. In particular embodiments, the aqueous solution includes a phosphate buffer solution. In particular embodiments, the aqueous solution includes a saturated potassium chloride solution.

(ii) Bubble Nucleation Conditions. Bubble nucleation can be carried out in an electrochemical cell apparatus that houses the aqueous solution and an electrode. An “electrode” refers to an electrical conductor. Electrodes are generally made of conducting materials, such as platinum, nickel, magnesium, aluminum, zinc, silver, gold, palladium, iridium, graphite carbon, indium oxide, tin oxide, mixed indium/tin oxide, stainless steel, mercury, or alloys and/or salts thereof. The term “alloy” refers to a combination of metals and/or other elements, or as would be understood by one of ordinary skill in the art. Iron or copper anodes may dissolve over time in solution. In particular embodiments, platinum anodes are preferred to limit the dissolving rate of the anode overtime. Particular embodiments utilize a platinum electrode, a nickel electrode, a gold electrode, a palladium electrode, a silver electrode, and/or a silver/silver chloride alloy electrode.

In particular embodiments, the electrode is a nanoelectrode. “Nano” refers to measurements of 10⁻⁹ in reference to a relevant measurement, such as a meter. In particular embodiments, the electrode is a nanoelectrode with a radius of 5-100 nm (e.g., 7, 8, 9, 10, 11, 12, 13, 14, 15, 16, 17, 18, 19, 20, 21, 22, 23, 24, 25, 26, 27, 30, 34, 45, 50, 57, 65, 70, 75, 80, 85, 90, 95, or 98 nm). The electrode's size can be dependent upon the size of the electrochemical cell or container in which one or more electrodes are housed. In particular embodiments, electrodes can be microelectrodes with radii dimensions of greater than 200 nm. During the gas evolution reaction, bubbles block the electrode's surface, thereby causing a decrease in current. Because of its smaller size, a nanoelectrode's surface area is more completely separated from the aqueous solution based on bubble formation. This leads to a sharper current drop after the formation of bubbles. The result is a current drop and clearer peak values. Therefore, in particular embodiments, a nanoelectrode is preferred.

For more information regarding electrodes, see AU2004206032B2 and CA2106205C.

As previously indicated, within systems and methods of the disclosure, a voltage is applied to the one or more electrodes within the aqueous solution to separate chemical species within the aqueous solution to generate gas bubbles such as hydrogen gas, oxygen gas, or nitrogen gas bubbles. Hydrogen evolution reactions (HER) are common gas evolution reactions that can be used to practice the systems and methods of the disclosure. When generating nitrogen gas, appropriate chemical precursors can be added to the aqueous solution. In particular embodiments, applied voltages can be less than 2 V. In particular embodiments, the applied voltage is between 2 V to 3 V.

During the application of the voltage in the gas evolution reaction, the current values should be monitored. Particular embodiments monitor the gas evolution reaction current using cyclic voltammograms. Particular embodiments negatively scan the electrode potential. Particular embodiments negatively scan the electrode potential at a constant rate. In particular embodiments, a potentiostat can apply a voltage to the electrode and measure the resulting current.

In particular embodiments, the peak current value and surfactant concentration, in solution, have a linear relationship. In particular embodiments, a predicted peak current value can be calculated for a surfactant using the formula:

iₚₑₐₖ=K_{H}4ₙFD_{H2}r[A(a log(C_{surfactant})+b)^{3/2}+P_{ambient}],

wherein K_{H} is Henry's law constant for H₂ gas; n is the number of electrons transferred per H₂; F is Faraday's constant; D_{H2} is the diffusion coefficient of H₂; r is the nanoelectrode radius; A is a constant (16π/3kT In(Z/J); a is the slope value of the linear relationship; log C_{surfactant} is the logarithmic concentration of the surfactant; b is the y-intercept value of the linear relationship; and P_{ambient} is the ambient pressure.

Particular embodiments include a database of peak current values, minimal current values, and/or differential values between the peak and minimal values associated with surfactants' concentrations in different aqueous solutions under different conditions. The database can include predicted values and/or experimentally derived values utilizing test samples. This database can provide a reference to compare an observed current value to deduce the identity and concentration of a detected surfactant. A processor can compare an observed value with the reference database's values and conditions to identify surfactants and the associated concentration and display the results of the comparison on a user interface display.

Thus, the systems and methods disclosed herein transduce changes in nucleation conditions based on the presence of a surfactant into an electrochemical signal that provides surfactant concentration.

Systems and methods described herein using peak current value obtained limits of detection (LOD) of 80 μg/L for PFOS and 30 μg/L for PFOA. The addition of a preconcentration step using solid-phase extraction brought the LOD for PFOS down to 40 ng/L. In particular embodiments, solid-phase extraction can be performed as in EPA method 537.

(iii) Aerosol-Based Preconcentration. An additional advance of the current disclosure provides a simple and efficient method to preconcentrate surfactants using electrochemical aerosol formation. In these embodiments, a voltage is applied to one or more electrodes within an aqueous solution to generate bubbles. This aspect of the disclosure takes advantage of the fact that surfactants accumulate in a solution at air-solution interfaces, such as at the interface between formed bubbles and the solution. When the bubbles burst at the aqueous solution's surface, a thin layer of liquid surrounding the bubble is ejected into the air. This liquid, which is high in surfactant concentration, can be captured and analyzed, utilizing various techniques.

In particular embodiments, electrodes can be provided as an anode and a cathode or a single electrode with both anodic and cathodic function. The anode is the positive electrode that electrons enter, resulting in oxidation, whereas the cathode is the negative electrode which electrons leave, resulting in reduction. Electrodes may be a foam electrode, foil electrode, rod electrode, or a plate. Foam electrodes have pores on their surface ranging from 0.05-0.1 mm. Particular embodiments include the use of a foil electrode as an anode or a cathode and a foam electrode as an anode or a cathode, or a combination thereof. The foil anode/cathode can have a size of 1 cm×1 cm, and the foam cathode can have a size of 1.5 cm×1.5 cm. In particular embodiments, the electrode's size depends on the size of the electrochemical cell or container which houses the electrode.

Within systems and methods of the disclosure, a voltage is applied to the one or more electrodes within the aqueous solution to separate chemical species within the aqueous solution to generate gas bubbles such as hydrogen gas, oxygen gas, or nitrogen gas bubbles. The preconcentration method generally utilizes a higher voltage to drive bubble formation than the earlier-described surfactant detection methods. In preferred embodiments, the voltage level for the preconcentration method is 30 volts. In particular embodiments, applied voltages can be between 2 V to 80 V for a period of time, such as 20 seconds to 10 minutes. During the time period, the applied voltage can be kept constant to generate a reproducible amount of bubble formation, although some variation in current can be acceptable.

Particular embodiments manipulate the parameters of the systems and methods to affect the size of created bubbles. The smaller the bubble size, the increase in preconcentration of the analyte on the bubble. The geometry of the bubble distribution size and radius can be obtained using computer simulation platforms. In particular embodiments, formed bubbles have a 70 μm radius. Methods to affect the size of formed bubbles are to adjust the electrode's depth under the surface of the aqueous solution, adjust the applied voltage, and/or to choose a foil electrode with a selected pore size.

In particular embodiments, the depth of the electrodes and the voltage applied can be manipulated to modulate the size of formed bubbles. For example, the depth of an electrode can be positioned between 10-40 cm below the aqueous solution's surface. In particular embodiments, the electrode can be placed 22, 23, 24, 25, 26, 27, 28, 29, 30, 31, or 32 cm below the aqueous solution's surface. The applied voltage can range from 15-100 volts. In particular embodiments, the applied voltage is 25, 30, 35, 40, 45, 50, 55, 60, 65, 70, 75, 80, or 85 V. A foil electrode with a smaller pore size creates smaller bubbles.

The time of voltage application can affect the amount of pre-concentrated samples that can be captured. In particular embodiments, liquid from the formed aerosol is captured at a rate of between 5-15 μL/min. In particular embodiments, liquid from the formed aerosol is captured at a rate of 9 μL/min. In particular embodiments, liquid from the formed aerosol is collected for a time period between 5 to 10 minutes.

As described herein, aerosol capture results in a preconcentrated sample that can have an enrichment factor between 500 to 1300-fold. The enrichment factor of the aerosol can increase with increasing carbon chain length of the surfactant. In particular embodiments, the enrichment factor is at least 1000-fold. In particular embodiments, the enrichment factor and bubble path length include a linear relationship.

(iv) Mass Spectrometry Analysis of Aerosol-Captured Samples. Mass spectrometry can be used to analyze samples enriched using aerosol capture. In particular embodiments, aerosol-enriched samples are diluted prior to mass spectrometry analysis to minimize the mass spectrometer's contamination. In particular embodiments, the aerosol solution is diluted 20 times before mass spectrometry analysis.

A diluted aerosol surfactant sample can be delivered to the analyzer by injecting or delivering the diluted sample into a sample inlet port located on the analyzer. During sample introduction, an interface may provide for continuous introduction of the sample to the ion source. Alternatively, the sample can be intermittently introduced to the ion source.

Suitable analyzers can be mass spectrometers; linear ion trap mass spectrometers; single-focusing mass spectrometers; static field mass spectrometers; dynamic field mass spectrometers; electrostatic analyzers; magnetic analyzers; quadrupole analyzers; time of flight analyzers (e.g., a matrix-assisted laser desorption/ionization (MALDI) Quadrupole time-of-flight mass spectrometer); electrospray-ionization, quadrupole time-of flight-tandem (ESI-Q-TOF) mass spectrometers; liquid chromatography-electrospray ionization-tandem mass spectrometers (LC-ESI MS/MS); fast atom bombardment (FAB) mass spectrometers; Wien analyzers; mass resonant analyzers; double-focusing analyzers; ion cyclotron resonance analyzers; ion trap analyzers; and/or combinations thereof in any order (e.g., as in a multi-analyzer system). Such analyzers are known in the art and are described in, for example, Introduction to Mass Spectrometry: Instrumentation, Applications, and Strategies for Data Interpretation, Edition 4, John Wiley & Sons, (2013)).

In particular embodiments, the diluted aerosol samples are analyzed by a linear ion trap mass spectrometer (LIT-MS). A LIT-MS is a quadrupole ion trap analyzer in which quadrupole rods confine ions radially (towards the detector). The static electrical potential on the end of the electrodes confine the ions axially. The LIT-MS is used as either an ion mass filter or as an ion trap by creating a potential well for the ions along the axis of the trap. The mass of the trapped ions is determined if the mass-to-charge (m/z) ratio of the ion lies between defined parameters.

In particular embodiments, the diluted aerosol samples are analyzed by a tandem MS system (an MS/MS system). An MS/MS system enables rapid analysis of low levels of analytes and can be used to maximize throughput. In particular embodiments, the analyzer includes an ionizing source for generating ions of a test surfactant and a detector for detecting the ions generated. The analyzer further includes a data system for analyzing mass data relating to the ions and deriving mass data relating to the test surfactant.

In one aspect, the ion source is an electrospray used to provide droplets to the analyzer, each droplet including a diluted amount of surfactant analyte. A high voltage is applied to a liquid stream during electrospray, causing large droplets to be subdivided into smaller and smaller droplets until a surfactant enters the gas phase as an ion. Ionization generally is accomplished when the surfactant loses or gains a proton. Ionization in electrospray is constant; MALDI can be used to achieve pulsed ionization. Other ionization methods include plasma desorption ionization, thermospray ionization, and fast atom bombardment ionization, as are known in the art. In particular embodiments, the analyzer includes an ion transfer section through which ions are delivered from the ion source to the detector. The ion transfer section includes an electric and/or magnetic field generator (e.g., an electrode ring) that modulates the acceleration of ions generated by the ionizing source. The electric/magnetic field generator directs ions through the ion transfer section of the analyzer to the ion detector. In particular embodiments, the analyzer includes an ion trap positioned between the ion transfer section of the analyzer and the detector for performing one or more operations such as ion storage, ion selection, and ion collision. The ion trap can be used to fragment ions produced by the ion source. The ion trap also can be used to store ions in stable orbits and to sequentially ejections based on their mass-to-charge values (m/z) to the detector. In particular embodiments, ions are trapped because of their electrostatic attraction to an inner electrode balanced with their inertia. When ions are in their desired orbit inside the ion trap, detection can be performed. In particular embodiments, the m/z ration detected is at a value of 269.0 m/z, 319.0 m/z, 369.0 m/z, 419.0 m/z, 469.0 m/z, 518.9 m/z, 568.9 m/z, 298.9 m/z, 398.9 m/z, and 498.9 m/z.

In particular embodiments, mass analysis is performed by the detector. In particular embodiments, the detector detects each ion's signal strength, which is a reflection of the amount of protonation of the ion. In particular embodiments, the detection sensitivity is within a range of 0.5 mg/L to 5 μg/L. In particular embodiments, the “Limit of Detection” (LOD) is a value of less than 40 ng/L in an aqueous solution. In particular embodiments, mass analysis is performed by the Fourier Transform mode, which measures coherent oscillations in the axial direction. In particular embodiments, mass analysis is performed by the mass selective instability mode, which involves ion ejection and collection onto a detector.

In particular embodiments, the linear ion trap mass spectrometer is operated in the negative ion detection mode with a spray voltage of ±4.0 kV, respectively. The solvent flow was set at 0.010 mL/min, and the data acquisition time was 30 s.

In particular embodiments, an additional separation section can be provided between the ion trap and detector to separate fragments generated in the ion trap (e.g., as in tandem MS).

In particular embodiments, the analyzer includes a data system for recording and processing information collected by the detector. The data system can respond to instructions from a processor in communication with the separation system and provide data to the processor. Preferably, the data system includes one or more of: a computer, an analog to digital conversion module; and control devices for data acquisition, recording, storage, and manipulation. In particular embodiments, the device includes a mechanism for data reduction, i.e., to transform the initial digital or analog representation of output from the analyzer into a form that is suitable for interpretation, such as a graphical display.

In particular embodiments, surfactant analysis is performed on each diluted aerosol droplet with a linear ion trap mass spectrometer (LTQ Velos, Thermo Fisher Scientific, San Jose, Calif., United States) mass spectrometer. In particular embodiments, solid-phase extraction (SPE) is used to preconcentrate the surfactant analyte. SPE is carried out by passing 1-Liter of the sample through the SPE cartridge at a rate of 10-15 mL/min.

(iv). Experimental Examples.

### Experimental Example 1. Bubble-Based Method for the Selective and Sensitive Electrochemical Detection of Surfactants

Abstract: The current disclosure presents the first bubble-based electrochemical method for the selective and sensitive detection of surfactants. The method takes advantage of the high surface activity of surfactant analyte to affect the electrochemical bubble nucleation. It then transduces the change in nucleation condition to an electrochemical signal for determining the surfactant concentration. Using this method, the present disclosure demonstrates the quantitation of perfluorinated surfactants in water, a group of emerging environmental contaminants, with a remarkable limit of detection (LOD) down to 30 μg/L and a linear dynamic range of over 3 orders of magnitude. With the addition of a preconcentration step, a LOD of 70 ng/L was achieved, which is the health advisory level for perfluorooctanesulfonate (PFOS) and perfluorooctanoic acid (PFOA) in drinking water established by the U.S. Environmental Protection Agency. The experimental results are in quantitative agreement with the theoretical model derived from classical nucleation theory. The method also exhibits an exceptional specificity for the surfactant analytes even in the presence of a 1000-fold excess of nonsurfactant interference. This method can be used to provide a universal electrochemical detector for surfactant analysis because of its simplicity and the surface-activity-based detection mechanism.

Introduction. Surfactants are widely used as dispersants, emulsifiers, detergents, fabric softeners, and wetting agents in many household items and industrial products and processes (Kronberg, et al., John Wiley & Sons Ltd., Chichester, 2003). Because of the environmental impact and toxicity of various surfactants, current legislation requires that the amount of surfactants released into the sewer system is minimized and that the concentrations in rivers and lakes are maintained at low levels (Zoller, et al., CRC Press: 2004, Vol. 121). For example, perfluorinated surfactants (PS) have been widely used in coating and surfactant applications since the 1950s (e.g., nonstick coating and fire-fighting foam) because of the chemical and thermal stability of a perfluoroalkyl moiety and its distinctive hydrophobic and lipophobic nature. As a result of the extensive use of PS and their emission, a broad range of these compounds have been detected in the environment, wildlife, and humans. Recent biomedical studies have revealed the positive associations between PS exposure and disease parameters in the general population (Grandjean, et al., New Solut. 2015, 25, 147-163). As a result, the U.S. Environmental Protection Agency identified addressing the problem of fluorinated substances as one of the national priorities in 2018. Many well-known methodologies for surfactant determination require either expensive and complicated instruments (for example, liquid and gas chromatography) or the use of relatively large amounts of organic solvents (such as chloroform in the spectroscopic “methylene blue” method) (Clesceri, et al., 20th ed.; American Public Health Association, 1999), making them unsuitable for in situ detection applications (Zhao, et al., Chem. Commun. 2019, 55, 1378-1381). Therefore, there is a critical need to develop new and improved methods for surfactant detection.

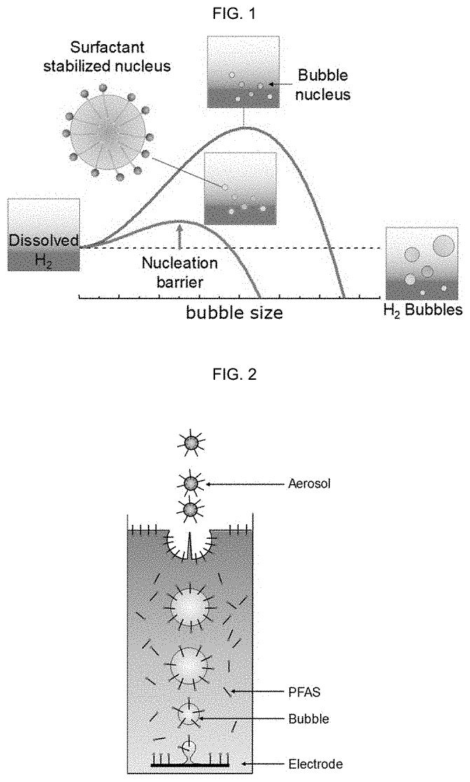

The formation and evolution of vapor and gas bubbles in a liquid body is a phenomenon of vast fundamental and applicative interest, for example, in commercial electrolytic processes (Xu, et al., Acc. Chem. Res. 2018, 51, 1590-1598; Chung, et al., Angew. Chem., Int. Ed. 2012, 51, 10089-10093), in cavitation, (Chung, et al., Angew. Chem., Int. Ed. 2012, 51, 10089-10093; Plesset, et al., Annu. Rev. Fluid Mech. 1977, 9, 145-185; Blake, et al., Annu. Rev. Fluid Mech. 1987, 19, 99-123), in biomedical applications (Zlitni, et al., Angew. Chem., Int. Ed. 2014, 53, 6459-6463; Chung, et al., Angew. Chem., Int. Ed. 2015, 54, 9890-9893), and in functional material fabrication (Zhang et al., Angew. Chem., Int. Ed. 2017, 56, 8191-8195; Pagano et al., Angew. Chem., Int. Ed. 2008, 47, 9900-9903; Drenckhan, et al., Angew. Chem., Int. Ed. 2009, 48, 5245-5247; Wang, et al., ACS Nano 2011, 5, 9927-9933). Here, the present disclosure presents a new application of gas bubbles for surfactant detection. The method is based on the interactions between gas nuclei and surfactant molecules during electrochemical gas bubble nucleation. According to classical nucleation theory (CNT) (Blander, et al., AIChE J. 1975, 21, 833-848), nucleation of a gas bubble requires a supersaturation of dissolved gas because of the energy barrier of establishing a new gas-liquid interface (FIG. 1). In the presence of surfactant molecules, gas nuclei can be stabilized because of the reduced surface tension of the gas-liquid interface, leading to a decrease in the supersaturation level required for bubble nucleation. In this disclosure, the high surface activity of surfactant analyte to affect the bubble nucleation and transduce the change in the supersaturation level required for bubble nucleation to electrochemical signal, for highly sensitive and specific detection of surfactant analytes, is utilized.

Experimental Section. Chemicals and Materials: Perchloric acid (HClO₄, 70%), sodium perchlorate (NaClO₄, 98%), tridecafluorohexane-1-sulfonic acid, nonafluorobutane-1-sulfonic acid, perfluorooctanoic acid, perfluoroheptanoic acid, undecafluorohexanoic acid, heptafluorobutyric acid, poly(ethylene glycol) (400 g/mol), TWEEN 20, lysozyme from chicken egg white, and humic acid were purchased from Sigma-Aldrich. Potassium perfluorooctanesulfonate was purchased from Matrix Scientific. Perfluoroheptanesulfonic acid was purchased from Synquest Laboratories. Glass capillary (outside diameter/inside diameter, 1.65/1.10 mm, soft temperature, 712° C.) was received from Dagan Corporation. Platinum wires (Pt wire, 25 μm diameter, 99.95%) were purchased from Surepure Chemetals. Silver conductive epoxy was purchased from MG Chemicals. A Visiprep SPE Vacuum manifold (Supelco Inc., Bellefonte, Pa., USA) was used for solid-phase extraction. BondElut LMS polymer 500 mg SPE cartridges were purchased from Agilent. Surface tension measurements were conducted using the pendant drop method on a Kruss DSA100 goniometer. All aqueous solutions were prepared from deionized (DI) water (PURELAB, 18.2 MΩ cm, total organic carbon <3 ppb).

Electrochemical Measurements. All experiments were carried out using a CHI 760E potentiostat and inside a grounded Faraday cage. A silver/silver chloride (Ag/AgCl) electrode in a saturated KCl solution was used as the counter/reference electrode during the measurements with nanoelectrodes. A mixture of 0.10 M NaClO₄ and 1.0 M HClO₄ was used as the supporting electrolyte for all of the experiments. A serial dilution of perfluorinated surfactants was made in 1.0 M HClO₄/0.10 M NaClO₄ solution. Cyclic voltammograms of nanoelectrodes were run to obtain the peak current for each compound with different concentrations. The scan rate was fixed at 100 mV/s.

Nanoelectrode Fabrication Method. According to a previously reported method, Pt nanoelectrodes were fabricated with some modifications (Zhang, et al., Anal. Chem. 2007, 79, 4778-4787). A 1.5 cm long Pt wire was attached to a tungsten rod using Ag conductive epoxy. The end of the Pt wire was electrochemically etched to make a sharp point in 15 wt % CaCl₂) solution. With the use of a function generator, a 110 Hz sinusoidal wave with an amplitude of 4.3 V was applied to the Pt wire for 60 s. The sharpened wire was washed with deionized water and was then inserted into a glass capillary and thermally sealed using an H₂—O₂ flame. The sealing was inspected against possible gas bubbles using an optical microscope during the sealing process. The sealed tip was then polished successively using silicon carbide polishing sandpapers (Buehler with grid size 600 and 1200) until a Pt nanodisk was exposed, which was monitored by an electronic feedback circuit. The radii of nanodisk electrodes, r, were determined by the diffusion-limited current for proton reduction (iₗᵢₘ) in 0.10 M HClO₄ solution containing 0.10 M NaClO₄. The migration effects are suppressed by adding 0.10 M NaClO₄ as the supporting electrolyte. The radii were calculated using the following equation: iₗᵢₘ=4nFDCr, where D is the diffusion coefficient of H⁺ and C is the concentration of HClO₄, respectively. A literature value of D=7.8×10⁻⁵ cm²/s was used (Chen, et al., J. Phys. Chem. C 2018, 122, 15421-15426). The radii estimated using this method are within 10% difference from the ones determined from the conventional ferrocene oxidation method.

Preconcentration Method. Sample preconcentration was carried out using solid-phase extraction following U.S. EPA Method 537. Briefly, solid-phase extraction cartridge cleanup, and conditioning was done with 15 mL of methanol followed by 18 mL of DI water. One liter of the sample was passed through the cartridge at a rate of 10-15 mL/min with the help of a vacuum manifold. Then the analyte was eluted from the cartridge with 15 mL of methanol. The eluate was collected and completely dried under a gentle stream of N₂ in a heated water bath (60-65° C.). Finally, 1.0 mL of 1.0 M HClO₄/0.10 M NaClO₄ solution was added to solvate the dried sample for electrochemical bubble-nucleation experiments.

Results and Discussion. To electrochemically probe the bubble-nucleation condition, the present disclosure adopted a nanoelectrode-based approach developed by Luo and White (Luo, et al., Langmuir 2013, 29, 11169-11175). In this approach, a sub-50-nm Pt nanoelectrode is used to perform a hydrogen evolution reaction (HER) in acid solutions. As the nanoelectrode potential is scanned negatively, the HER current increases exponentially until it reaches a peak value (iₚₑₐₖ). Past iₚₑₐₖ, the HER current immediately drops to a minimal value, which corresponds to the nucleation and formation of a gas bubble at the nanoelectrode, blocking the electrode surface (Luo, et al., Langmuir 2013, 29, 11169-11175; Chen, et al., Langmuir 2015, 31, 4573-4581; Chen, et al., J. Phys. Chem. Lett. 2014, 5, 3539-3544). The supersaturation level of dissolved H₂ gas required for H₂ bubble nucleation is proportional to the iₚₑₐₖ value (Luo, et al., Langmuir 2013, 29, 11169-11175).

Perfluorooctanesulfonate (PFOS) and perfluorooctanoate (PFOA) were chosen as the model analytes because they have been found at the highest frequency and concentration in the environment and humans among all PS. The PS pattern in global river waters reveals that PFOS and PFOA account for 60% of the total mass concentration of PS (Huset, et al., Environ. Sci. Technol. 2008, 42, 6369-6377; Loos, et al., Environ. Pollut. 2009, 157, 561-568; McLachlan, et al., Environ. Sci. Technol. 2007, 41, 7260-7265; Nakayama, et al., Environ. Sci. Technol. 2007, 41, 5271-5276; Murakami, et al., Environ. Sci. Technol. 2008, 42, 6566-6572; So, et al., Chemosphere 2007, 68, 2085-2095; Yeung et al., Chemosphere 2009, 76, 55-62). This percentage is up to >80% in biological samples such as human milk and serum because of the bioaccumulation of PFOA and PFOS (Karrman, et al., Environ. Health Perspect. 2007, 115, 226-230). FIG. 3A shows the cyclic voltammograms of an 11 nm radius Pt nanoelectrode in PFOS-containing HClO₄ solutions. All voltammograms at various concentrations of PFOS (C_{PFOS}) exhibited a cathodic peak at ca. −0.3 V, corresponding to the H₂ bubble nucleation and formation at the nanoelectrode surface. The C_{PFOS} was varied from 10⁻⁴ to 10⁻¹ g/L. As C_{PFOS} increases, iₚₑₐₖ decreases. When iₚₑₐₖ is plotted against log(C_{PFOS}), there is a good linear relationship between them (R²=0.92) with a slope of −0.82 nA/dec (FIG. 3B). The LOD based on 3 times the standard deviation of the blank (i.e., in the absence of PFOS) is calculated to be 80 μg/L. The reduced iₚₑₐₖ in response to the increasing PFOS concentration is consistent with the detection mechanism that PFOS stabilizes bubble nuclei and, therefore, lowers the supersaturation requirement for bubble nucleation.

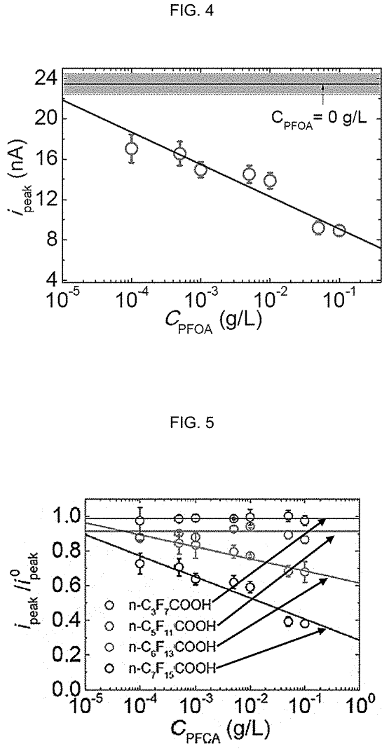

The same linear response has also been observed for PFOA, the other dominant PS contaminant, and the carboxylic acid counterpart of PFOS, in the same concentration range (FIG. 4). The obtained LOD for PFOA is 30 μg/L, which is slightly better than that for PFOS. It should be caused by the higher surface activity of PFOA than PFOS (their corresponding surface tension minima in water are 15.2 and 34.5 dyn/cm, respectively) (Shinoda, et al., J. Phys. Chem. 1972, 76, 909-914). The present disclosure's LODs, when utilizing bubble-nucleation for the detection of PFOA and PFOS, are 2 orders of magnitude better than those of suppressed conductivity detection (2 mg/L) (Hori, et al., Chemosphere 2004, 57, 273-282) and slightly worse than those of tandem mass spectrometry detection (0.5 μg/L), the two most common detection methods for surfactant analysis used in high-performance liquid chromatography.

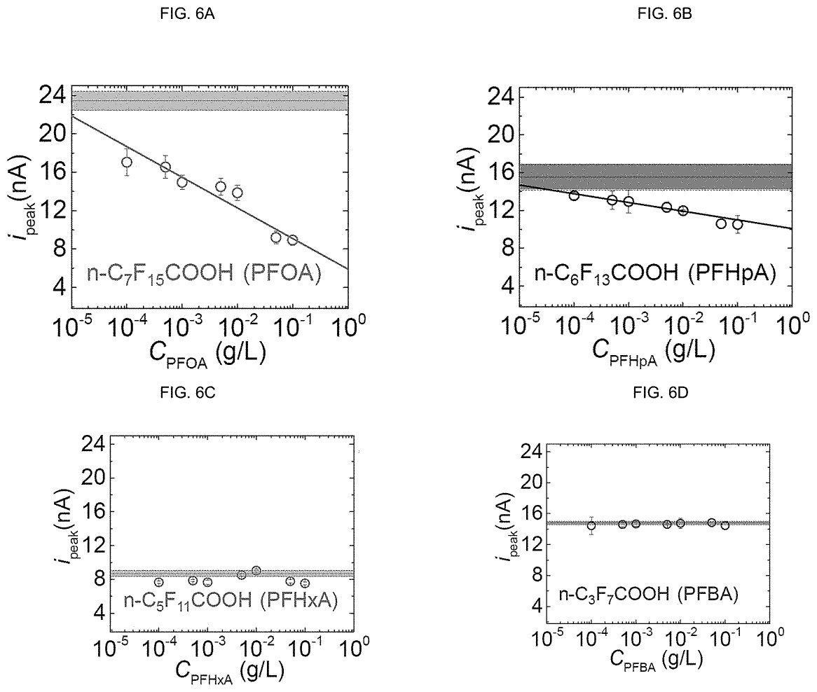

The PS compounds were further tested with different fluoroalkyl chain lengths using the described methods. FIG. 5 shows the peak current plot against the concentration of perfluorinated carboxylic acids (PFCA) with fluoroalkyl chain length, n=3, 5, 6, and 7. The peak currents are normalized with respect to the peak current in the absence of PFCA to account for the nanoelectrode size effect as larger electrodes require larger currents to nucleate a bubble (Chen, et al., J. Phys. Chem. C 2018, 122, 15421-15426; Chen, et al., J. Phys. Chem. Lett. 2014, 5, 3539-3544; Chen, et al., Langmuir 2018, 34, 4554-4559). The corresponding unnormalized data are provided in FIG. 6A-6D. As n decreases from 7 to 3, the slope is reduced from −0.12 dec⁻¹ at n=7 to −0.07 dec⁻¹ at n=6 and becomes close to 0 when n=5 and 3.

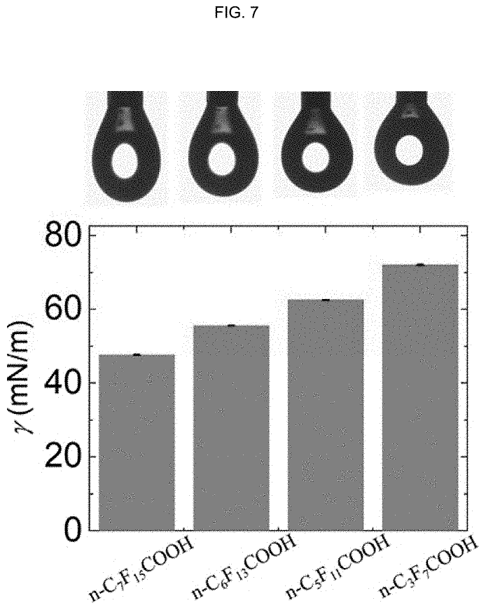

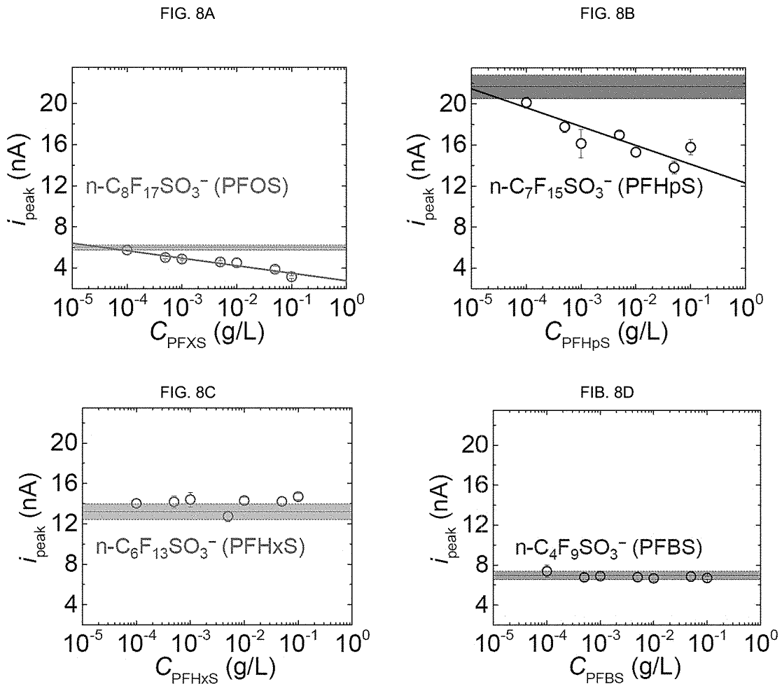

The trend of sensitivity change is consistent with the order of surface activity: n-C₇F₁₅COOH>n-C₆F₁₃COOH>n-C₅F₁₁COOH>n-C₃F₇COOH (FIG. 7), further confirming the mechanism in FIG. 1. A similar trend has also been observed for perfluoroalkyl sulfonate compounds (FIG. 8A-8D).

To quantitatively understand the detector response, the expression of iₚₑₐₖ as a function of C_{PFOS} was derived. According to CNT, the formation free energy of a gas bubble in solution, ΔG_{bubble}, is the sum of the energy cost of creating a new gas/liquid interface and the energy gain through the liberation of dissolved gas into the bubble volume, as expressed by Eq 1 (Talanquer et al., J. Chem. Phys. 1995, 102, 2156-2164).

$$Δ {G}_{bubble} = 4 π γ {r}_{bubble}^{2} + \frac{4 π}{3} Δ {G}_{V} {r}_{bubble}^{3} \qquad Eq . 1$$

where γ is the surface tension of the gas/liquid interface, and ΔG_{V} is the energy difference between the dissolved and gaseous state of the molecule in that volume. ΔG_{bubble} initially increases as a function of r_{bubble} before reaching a peak value, E_{nuc}=16πγ3/3 (ΔG_{V})², which is the nucleation energy barrier depicted in FIG. 1. Bubbles that overcome this energy barrier are energetically favored to continue to grow; otherwise, they are inclined to shrink and return to the dissolved form. Because bubbles of the critical size necessarily arise from the growth of subcritical nuclei, their formation relies upon relatively improbable fluctuations along the free energy barrier. The rate of critical nuclei formation or nucleation rate, J, is thus governed by the Arrhenius equation:

$$J = Z exp ( - \frac{{E}_{nuc}}{kT} ) = Z exp ( - \frac{16 π {γ}^{3}}{3 Δ {G}_{V}^{2} kT} ) \qquad Eq . 2$$

In the described experiment, the potential of a nano-electrode negatively at a constant scan rate (that is, a fixed duration time at each potential) was scanned to nucleate an H₂ gas bubble, and then the iₚₑₐₖ was recorded. Because the time required to nucleate a bubble defines the nucleation rate (J) when the duration time is fixed, a threshold value was set for J, and the minimum current to reach this value was sought. Hence, Eq. 2 can be rearranged and simplified to be:

ΔG_{V,nuc}=Aγ^{3/2} Eq. 3:

where A is a constant (=(16 π/3kT In(Z/J)) and ΔG_{V, nuc} is the volume energy difference of the gas molecules when a bubble nucleates.

On the left side of Eq. 3, ΔG_{V, nuc} can be expressed as a function of iₚₑₐₖ (German, et al., J. Am. Chem. Soc. 2018, 140, 4047-4053; German, et al., Faraday Discuss. 2016, 193, 223-240):

$$Δ {G}_{V , nuc} = \frac{{i}_{peak}}{{K}_{H} 4 {nFD}_{{H}_{2}} r} - {P}_{ambient} \qquad Eq . 4$$

where K_{H} is Henry's law constant for H₂ gas, D_{H2} is the diffusion coefficient of H₂, n is the number of electrons transferred per H₂ (=2), F is Faraday's constant, r is the nanoelectrode radius, and P_{ambient} is the ambient pressure.

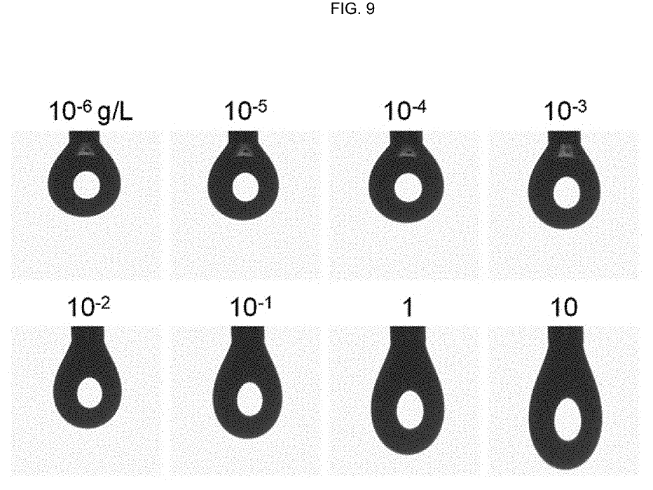

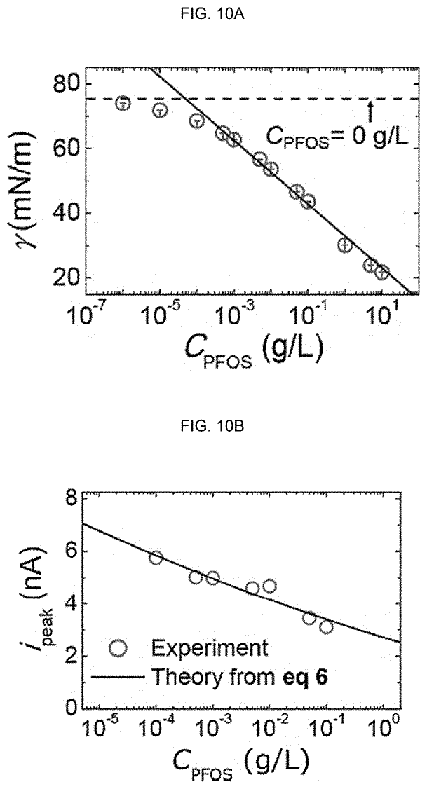

On the right side of Eq. 3, γ is a nonlinear function of C_{PFOS} governed by the Gibbs equation (Menger, et al., Langmuir 2011, 27, 13975-13977). They of the PFOS-containing solutions was measured by the pendant drop method (FIG. 9). The plot of γ versus log(C_{PFOS}) in FIG. 10A reveals an excellent linear relationship at the concentration range from 10⁻⁴ to 10 g/L. Outside this range, the data starts deviating from the linearity. Accordingly, γ can be numerically expressed by:

γ=a log(C_{PFOS})+b Eq. 5:

with a=−9.8 and b=33 for C_{PFOS}=10⁻⁴ to 10 g/L. The linear function intercepts with they value of the blank (C_{PFOS}=0) at C_{PFOS}=50 μg/L, which is consistent with the experimental LOD of 80 μg/L for PFOS. Substituting Eq 4 and Eq 5 into Eq 3, the following expression of iₚₑₐₖ was obtained:

iₚₑₐₖ=K_{H}4ₙFD_{H2}r[A(a log(C_{PFOS})+b)^{3/2}+P_{ambient}]. Eq. 6:

The experimental data agree very well with the theoretical fit in the form of Eq 6 (FIG. 10B), which again confirms the proposed bubble-based detection mechanism. The above derivation concluded that the nearly linear relationship between iₚₑₐₖ and log(C_{PFOS}) originates from the linear dependence of the γ on log(C_{PFOS}). Therefore, the sensitivity of this detection method is determined by the surface activity of analytes. Additionally, Eq. 6 also predicts that the electrode size (r) and properties of electrogenerated gas (D_{H2}, K_{H}, and n) will contribute to the sensitivity of this method.

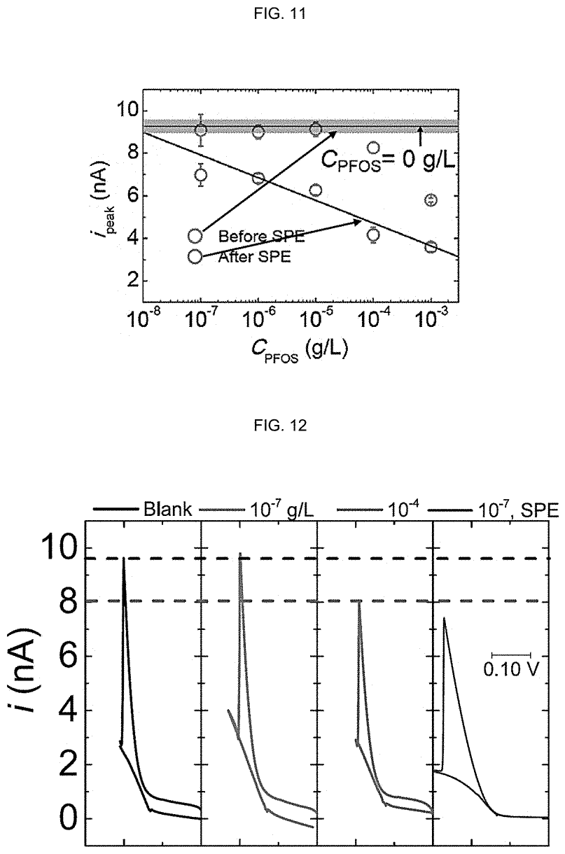

The native LOD of the detection method is 30 and 80 μg/L for PFOA and PFOS, respectively, which is limited by the surface activity of these two compounds. These values are 3 orders of magnitude higher than the desired LOD: 70 ng/L, which is the health advisory for PFOS and PFOA in drinking water established by the U.S. EPA. This challenge can be overcome by adding a preconcentration step using solid-phase extraction, which is currently used in the standard U.S. EPA method for PS analysis. FIG. 11 shows the LOD for PFOS was improved to 40 ng/L after a 1000-fold preconcentration step using solid-phase extraction. The corresponding cyclic voltammograms are provided in FIG. 12.

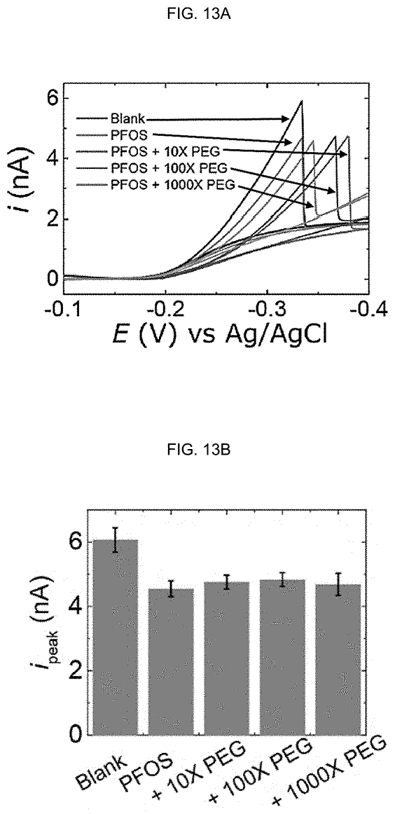

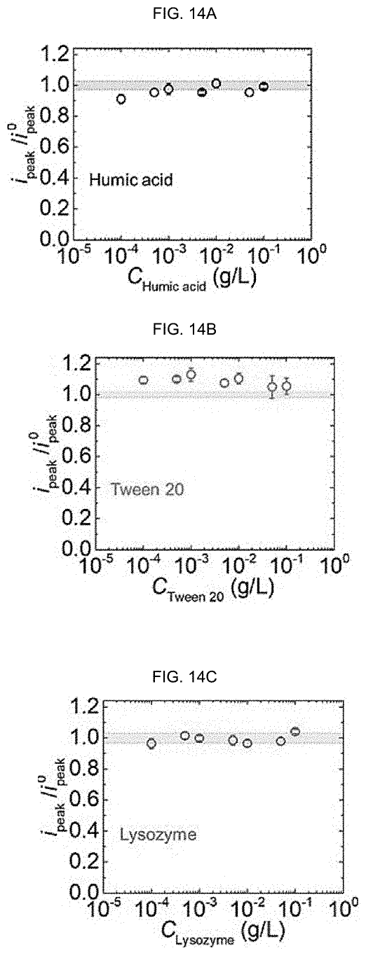

The specificity of the described method was further tested for detecting surfactant analytes by adding an excess of nonsurfactant interference, poly(ethylene glycol) (PEG, 400 g/mol), which has a similar molecular weight as PFOS. FIG. 13A shows the cyclic voltammograms of a Pt nanoelectrode in the presence of 1 mg/L PFOS and a 10-, 100-, and 1000-fold excess of PEG. The addition of PEG leads to a negative shift of the HER onset potential as compared to the PFOS-only sample, but the iₚₑₐₖ does not show any notable difference (FIG. 13B). Different concentrations of humic acid (FIG. 14A) and lysozyme (FIG. 14B) were also tested. No trend in the peak current was observed than that of the blank (FIGS. 14A, 14B). These results show the exceptional specificity of the method for surfactant analytes. However, the peak current change for a neutral surfactant, Tween-20, was not observed (FIG. 14C). The reason for this unusual behavior is currently under investigation.

In conclusion, a bubble-based electrochemical detection method for surfactant analysis has been presented for the first time. This method has a high specificity for surfactant analytes, a broad linear dynamic range of over 3 orders of magnitude, and a remarkable LOD of 30 μg/L (2 orders of magnitude better than suppressed conductivity detection, a conventional detection method for surfactant analysis). With a preconcentration step, the present disclosure demonstrated the improvement of the LOD for PFOS to the target LOD. The theory for this new method was established. This method can be used as a universal electrochemical detector for surfactant analysis.

### Experimental Example 2. 1000-Fold Preconcentration of Per- and Polyfluorinated Alkyl Substances within 10 Minutes Via Electrochemical Aerosol Formation

Abstract: The current disclosure presents a simple and efficient method for preconcentrating per- and polyfluorinated alkyl substances (PFAS) in water. This method was inspired by sea-spray aerosol enrichment in nature. Gas bubbles in the ocean serve to scavenge surface-active material, carrying it to the air-ocean interface, where the bubbles burst and form a sea-spray aerosol. These aerosol particles are enriched in surface-active organic compounds such as free fatty acids and anionic surfactants. In this method, H₂ microbubbles were in situ generated by electrochemical water reduction using a porous Ni foam electrode. These H₂ bubbles pick up PFAS as they rise through the water column that contains low concentration PFAS. When these bubbles reach the water surface, they burst and produce aerosol droplets enriched in PFAS. Using this method, the current disclosure demonstrates 1000-fold preconcentration for ten common PFAS in the concentration range from 1 pM to 1 nM (or 0.5 ng/L to 500 ng/L) in 10 min. A diffusion-limited adsorption model that is in quantitative agreement with experimental data was also developed. This method was also demonstrated to preconcentrate PFAS in tap water, indicating its use for quantitative analysis of PFAS in real-world water samples.

Experimental Section. Chemicals and Materials: Perfluorohexanoic acid (PFHxA, 97%), perfluoroheptanoic acid (PFHpA, 99%), perfluorooctanoic acid (PFOA, 95%), perfluorononanoic acid (PFNA, 97%), perfluorodecanoic acid (PFDA, 98%), perfluoroundecanoic acid (PFUnDA, 95%), perfluorododecanoic acid (PFDoDA, 95%), perfluorobutanesulfonic acid (PFBS, 98%), perfluorohexanesulfonic acid (PFHxS, 98%), perfluorooctanesulfonic acid (PFOS, 98%), sodium phosphate dibasic (99%), sodium phosphate monobasic (99%), methanol (99.9%), and nickel foam (mean aperture: 0.074 mm) were purchased from Sigma-Aldrich. Platinum electrodes were purchased from Aida Hengsheng (Tian Jin, China). Glass slides were purchased from Fisher Scientific. Deionized water (PURELAB, 18.2 MΩ cm, total organic carbon <3 ppb) was used in all of the experimental processes.

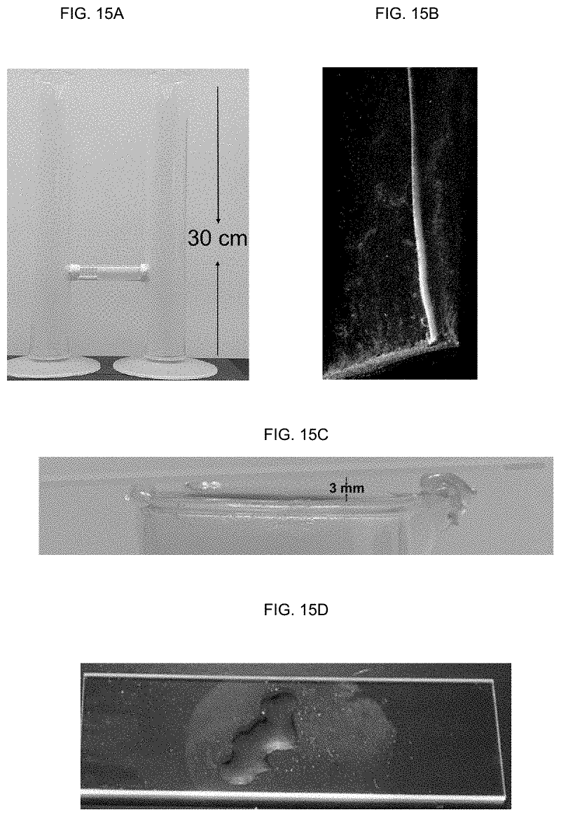

Electrochemical Aerosol Enrichment. All the enrichment experiments were carried out in a home-built H-type two-compartment electrochemical cell (FIG. 15A-15D). The cell was filled with 700 mL of 0.2 M phosphate buffer solution (pH=7.0). A 1 cm×1 cm Pt foil and a 1.5 cm×1.5 cm Ni foam electrode were separately immersed in the two compartments and used as the anode and cathode, respectively. A constant voltage was applied between the two electrodes to electrolyze water. H₂ and O₂ bubbles were formed at the Ni electrode and Pt electrode, respectively. The aerosol produced by bubble bursting was collected using a slanted glass slide placed at 3 mm above the liquid surface (FIG. 15C). Twenty μL of the collected aerosol was transferred to a polypropylene microcentrifuge tube from the glass slide using a pipet.

Mass Spectrometry Analysis. Before analysis, the collected aerosols were diluted 20 times in 50:50 v:v H₂O/MeOH to ease sample handling and to minimize chemical contamination of the mass spectrometer. Collected aerosols were analyzed operating a linear ion trap mass spectrometer (LTQ Velos, Thermo Fisher Scientific, San Jose, Calif., United States) (El-Baba, et al., Rapid Commun. Mass Spectrom. 2014, 28, 1175-1184) with electrospray ionization. Mass spectra were acquired in the negative ion detection mode with a spray voltage of ±4.0 kV, respectively. The solvent flow was set at mL/min, and data acquisitions time was 30 s. The transfer capillary was heated to 275° C.

Bubble Size Analysis. Photographs of gas bubbles in the solution were taken using a Sony RX100 M4 (full manual mode, aperture f8, ISO 800, shutter speed 1/2000- 1/1000 s) and analyzed using ImageJ software (NIH, Bethesda, Md.) to obtain the bubble size distributions.

Finite Element Simulation. The finite element simulations were performed using COMSOL Multiphysics 5.3 (Comsol, Inc.) on a high-performance desktop PC. The simulation geometry, mesh, and boundary conditions are provided below in the section entitled “Supporting Information.”

Tap Water Samples. Tap water was collected at the chemistry building of Wayne State University. No PFAS was found in the tap water sample. A mixture of PFAS was added to the tap water to prepare an artificial PFAS-contaminated tap water, which contained 0.04 nM (16 ng/L) PFHxS, 0.02 nM (8 ng/L) PFOA, and 0.2 nM (100 ng/L) PFOS. The same procedure was used to preconcentrate PFAS as described above.

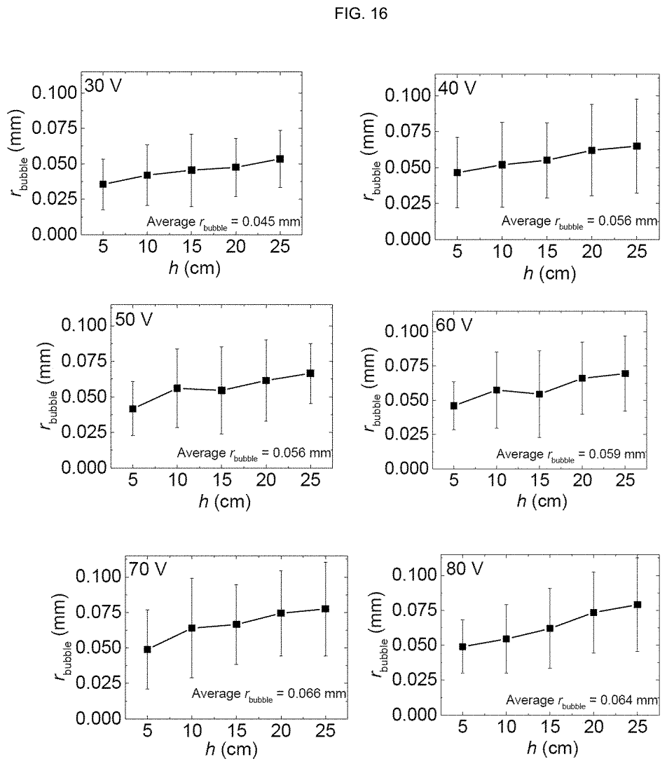

Results and Discussion. Preconcentration of PFAS: Preconcentration of PFAS. The study was carried out using perfluorooctanesulfonic acid (PFOS) as the model analyte. It is one of the PFASs found most frequently and at the highest concentration in the environment and humans (Huset, et al., Environ. Sci. Technol. 2008, 42, 6369-6377; Loos, et al., Environ. Pollut. 2009, 157, 561-568; Murakami, et al., Environ. Sci. Technol. 2008, 42, 6566-6572; Karrman, et al., Environ. Health Perspect. 2007, 115, 226-230). A Ni foam with nominal aperture sizes of 0.074 mm was used as the cathode, a Pt foil as the anode, and 10⁻⁸ M (or 5 μg/L) PFOS in 0.2 M phosphate buffer (pH=7.0) as the electrolyte. The Ni foam electrode was immersed at a depth of 25 cm. At a voltage of 70 V, the current level was 0.8 A, which corresponds to a volumetric flux of 5 mL of H₂ per minute. The radii of H₂ bubbles slightly increased from 0.05 mm to 0.08 mm, with an average value of 0.07 mm as they rose to the water surface (FIG. 16). The average bubble velocity was 1.2 cm/s. The aerosol droplets formed by bubble bursting were collected at a glass slide at 3 mm above the water surface (FIG. 15C). The collection rate of the aerosol droplets was estimated as 9 μL/min. After 5 min aerosol collection, millimeter-sized droplets were visible on the glass slide (FIG. 15D). To avoid chemical contamination of the mass spectrometer because of the high PFOS concentration in these droplets, dilution was done 20 times using a 50/50 v/v H₂O and MeOH mixture before mass spectrometry analysis.

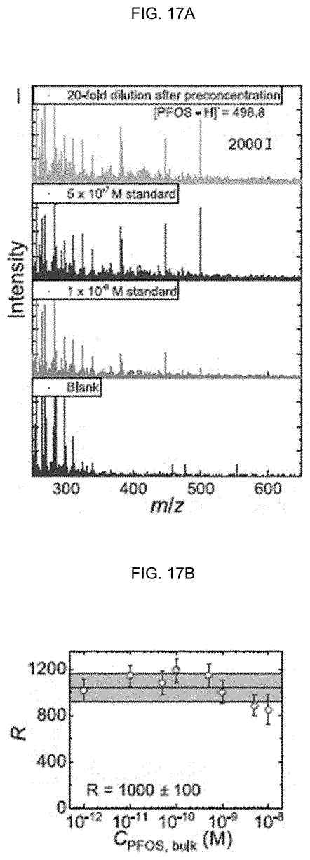

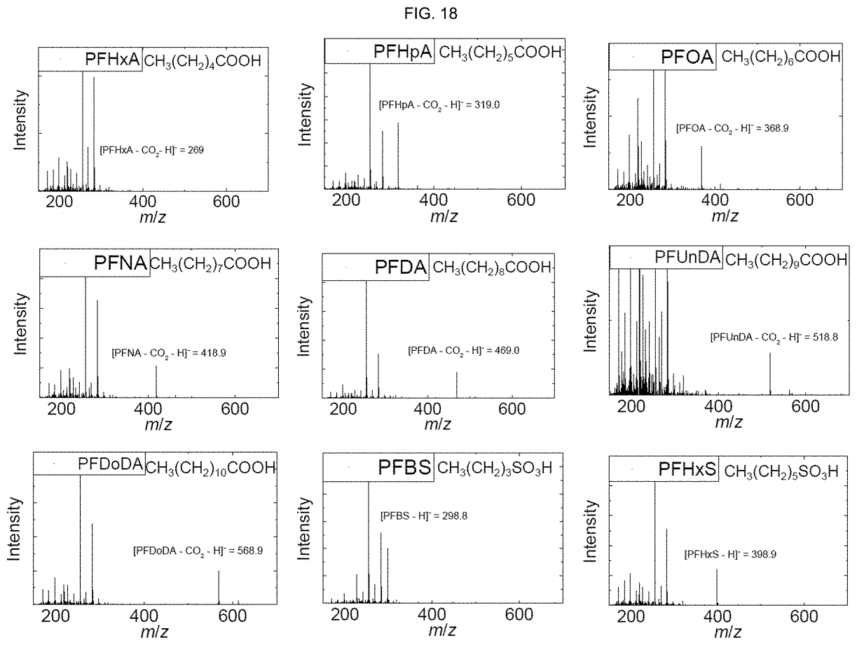

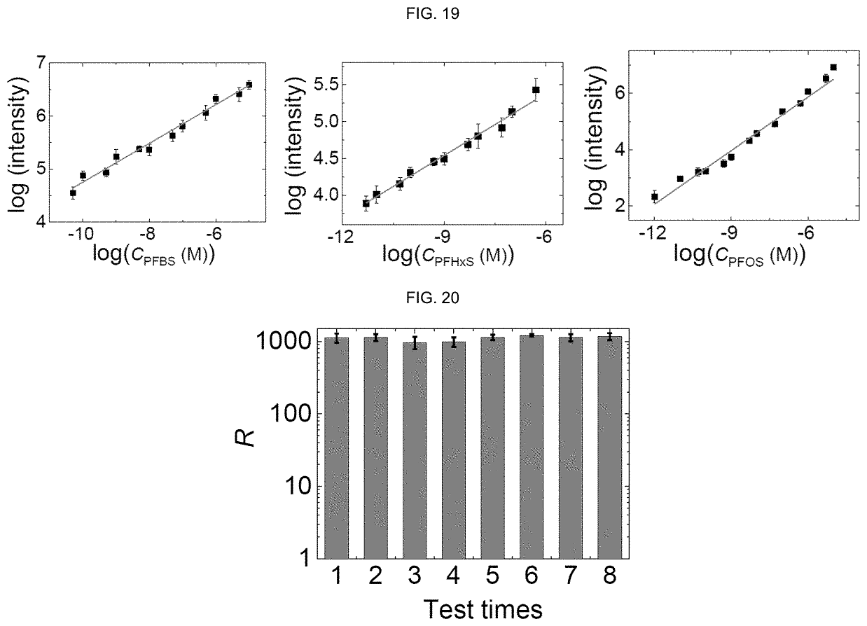

FIG. 17A shows the mass spectra of diluted aerosol sample, two PFOS standards (5×10⁻⁷ M and 10⁻⁸ M both in 50/50 v/v H₂O and MeOH mixture), and the blank in the negative ion detection mode. These mass spectra were recorded and accumulated continuously throughout the acquisition of each sample for 30 s. The signal at mass-to-charge (m/z)=498.8 corresponds to [PFOS-H]⁻. Good linearity was found between the signal intensity at m/z=498.8 and the PFOS concentration in standard solutions ranging from 10⁻¹² to 10⁻⁵ M (or 0.5 ng/L to 5 mg/L) with an r² value of 0.98 (FIGS. 18 and 19). Therefore, the signal intensity of [PFOS-H]⁻ was directly used to quantify the PFOS in all collected aerosol samples. The diluted aerosol sample shows a similar concentration as the 5×10⁻⁷M standard, indicating an enrichment factor of 1000-fold using the electrochemical aerosol formation method. The control experiments using 10⁻⁸ M standard and the blank show very low intensity at m/z=498.8. Other PFOS solutions with a concentration between 10⁻¹² M and 10⁻⁸ M (or 0.5 ng/L to 5 μg/L) were further tested using the preconcentration method. At C_{PFOS, bulk}<10⁻⁹ M (or 500 ng/L), R is around 1000. At higher concentrations, R starts to decrease to 800. Overall, the preconcentration method achieved an average R of 1000±100 for PFOS. To test the method's reproducibility, eight independent enrichment experiments were carried out following the same experimental protocol, and the enrichment factors were measured. A between-run coefficient of variation of 8% (FIG. 20) was obtained, indicating good producibility of the method.

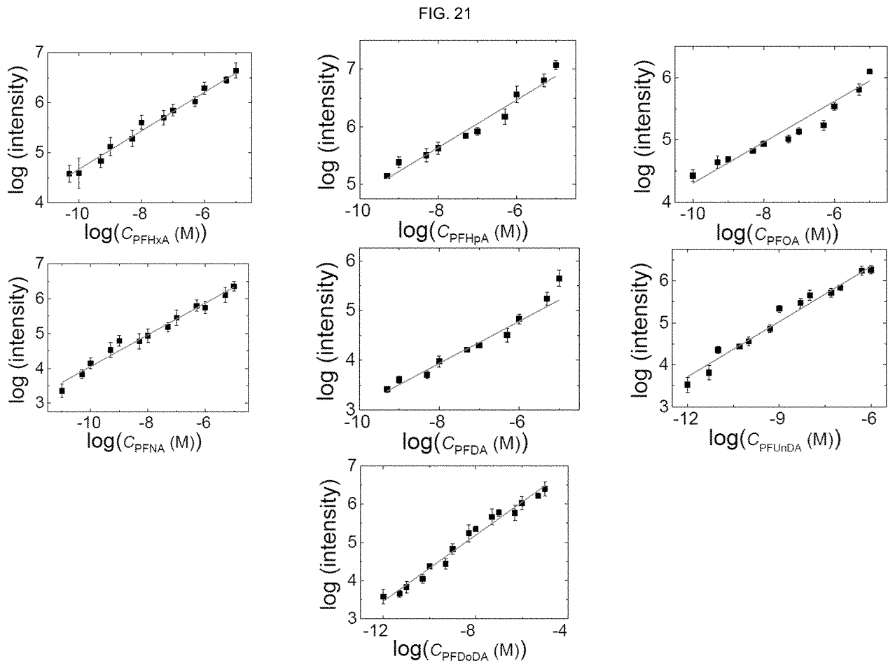

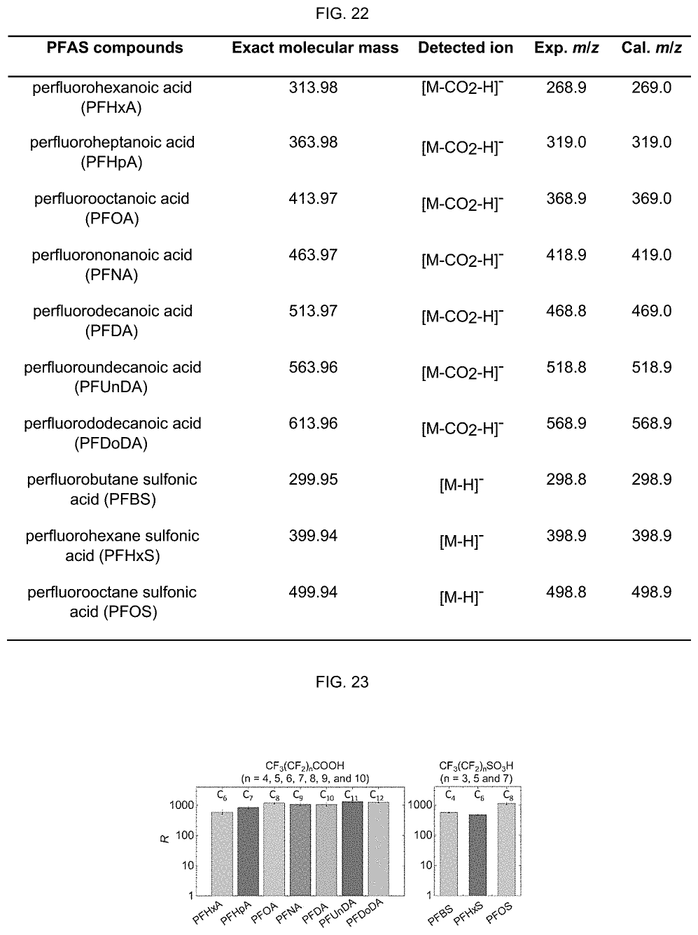

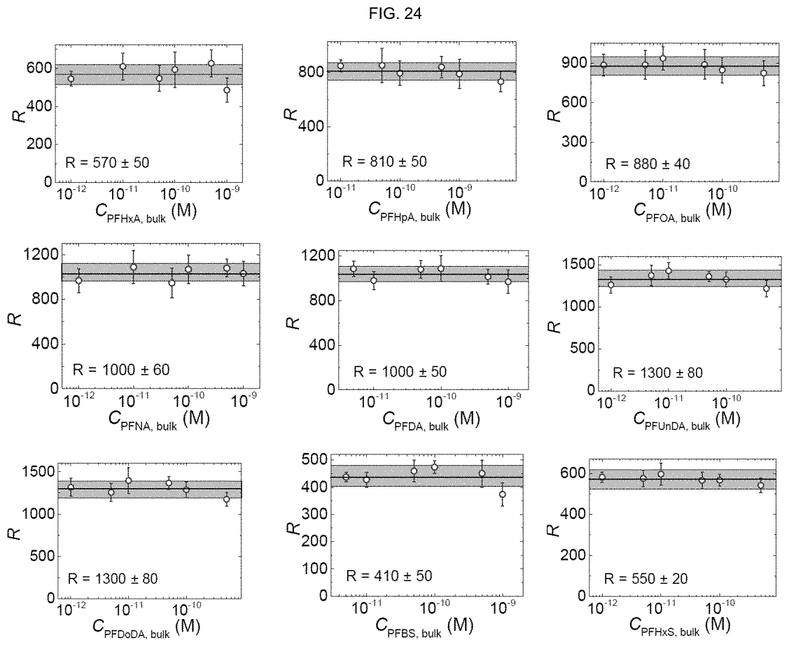

Besides PFOS, other 9 common PFAS, including 7 perfluorinated carboxylic acids with carbon chain lengths from 6 to 12 (PFHxA, PFHpA, PFOA, PFNA, PFDA, PFUnDA, and PFDoDA) and 2 perfluorinated sulfonic acids (PFBS and PFHxS), were also tested. The preconcentration experiments were conducted using a Ni foam electrode at an immersion depth of 25 cm and a voltage of 70 V for 10 min, and R was measured using mass spectrometry. The information on the ion peaks used for quantitation is provided in FIGS. 18, 19, 21, and 22. FIG. 23 shows the R's for all 10 PFAS are close to 1000 at CPFAS, bulk=10⁻¹⁰ M. The R slightly increases with the chain length of PFAS from 500 to 1300, which is probably caused by the increased surface activity. Most importantly, for each PFAS, the R-value varied by <10% at the concentration range of 10⁻¹² to 10⁻⁹ M (FIG. 24). The consistent enrichment performance of the method at a large concentration range makes it a technique useful in real-world applications.

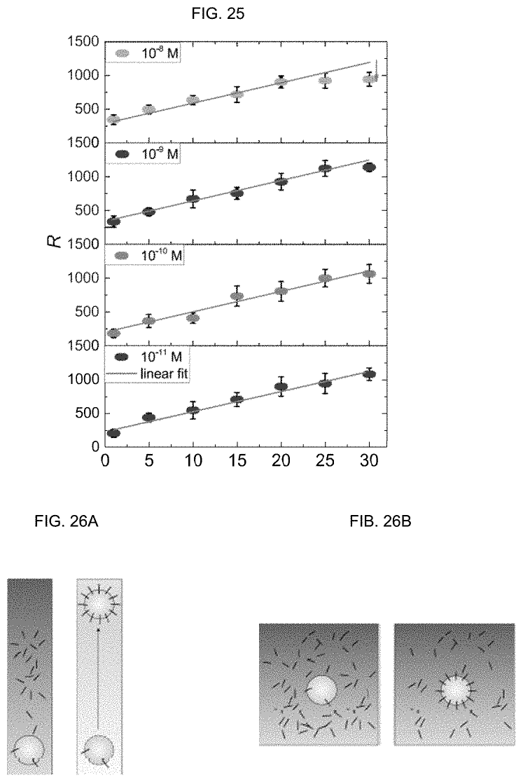

Preconcentration Mechanism. To understand the preconcentration mechanism, a bubble path length (h) dependence study was conducted. The Ni foam electrode was placed at different depths of the electrolyte solution to control the interaction time between PFOS and H₂ bubbles. No significant changes in current at different h (i=0.74±0.08 A) was observed when a constant voltage of 70 V was applied. FIG. 25 shows R vs. h at different C_{PFOS, bulk}. At C_{PFOS, bulk}=10⁻¹¹ M or 5 ng/L, there is a great linear relationship between R and h with a slope of 30 cm⁻¹ and r²=0.99. At C_{PFOS, bulk}=10⁻¹⁰ M and 10⁻⁹ M, the same linearity and slope were found. Until C_{PFOS, bulk} was increased to 10⁻⁸ M, the R-value started deviating from the linearity and reached a plateau at h=25 and 30 cm, suggesting the adsorption reached an equilibrium.

To quantitatively understand the linearity in FIG. 25, a model previously developed by Chen and co-workers (Chingin, et al., ACS Omega 2018, 3, 8709-8717) for rhodamine dyes adsorption onto the surface of a rising spherical bubble was first revisited. In the Chen model, it was assumed (1) the liquid near a bubble was renewed constantly due to bubble movement and (2) every surfactant that meets a rising bubble got adsorbed (the “total adsorption” model in FIG. 26A). Based on these two assumptions, the total amount of surfactants adsorbed by a bubble (n) was expressed as the product of the solution volume that a bubble sweeps and the bulk concentration of surfactant (C_{bulk})

n=πr²_{bubble}hC_{bulk} Eq. 7:

where r_{bubble} is the bubble radius. The surfactant concentration in the aerosol, Cₐₑᵣₒₛₒₗ, was then estimated to be:

Cₐₑᵣₒₛₒₗ≈150C_{bulk}h/r_{bubble} Eq. 8:

by relating the total volume of aerosol droplets produced by a single bubble to the bubble size (Brasz, et al., Phys. Rev. Fluids 2018, 3, 074001). Using Eq 8 and the present disclosure's experimental conditions, the enrichment rate (∂R/∂h) was calculated to be 2.1×10⁴ cm⁻¹, which is 700 times the current disclosure's experimental result of 30 cm⁻¹.

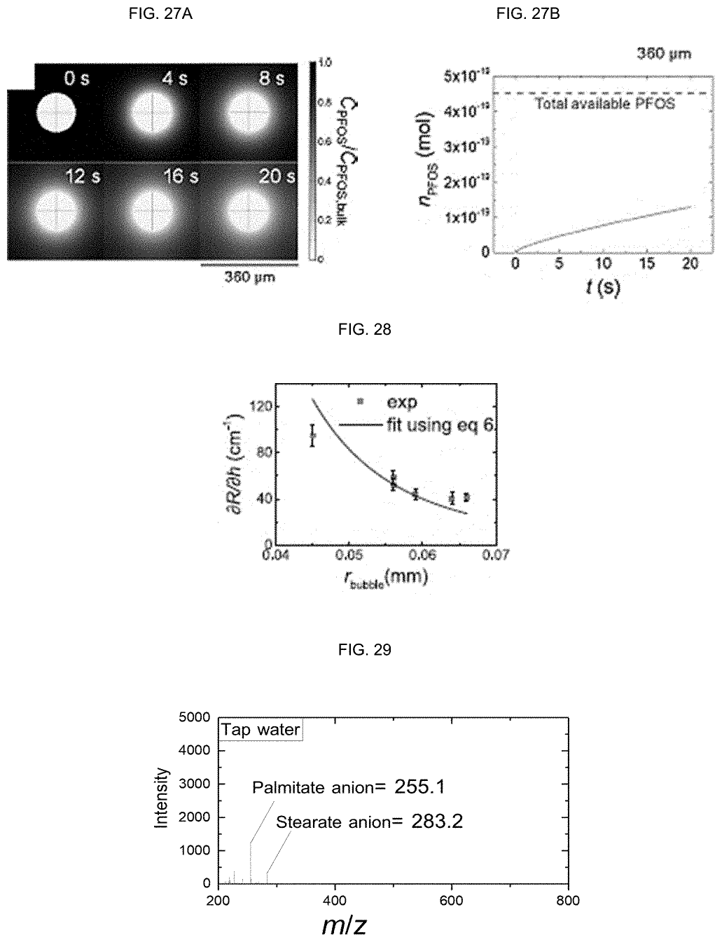

To address this discrepancy, a simple diffusion-limited adsorption model (FIG. 26B) was proposed. In the present disclosure's model, the liquid near a bubble is not renewed and moves at the same velocity as the rising bubble. Because PFOS has very high surface activity (Schultz, et al., Environ. Eng. Sci. 2003, 20, 487-501) and the bubble surface is far from being saturated with PFOS during its lifetime, it was assumed the bubble surface acted as a PFOS sink. Therefore, the adsorption of PFOS is limited by the diffusion of PFOS from the surrounding solution to the bubble surface. It was estimated that there were 106 H₂ bubbles in the bubble stream above the Ni foam electrode from electrochemical current and bubble velocity. The mean interbubble distance is, therefore, 360 μm. The “PFOS source” for a 70-μm-radius bubble is a solution cube with a side length of 360 μm. Note that the size of a rising bubble was gradually increasing, so the average bubble radius of 70 μm for simplicity was used. Using these parameters, the diffusion of PFOS to bubble surface, during the 20 s lifetime of a bubble, was simulated using a finite element simulation (see section “Supporting Information” below). FIGS. 27A and 27B show the simulated C_{PFOS}/C_{PFOS, bulk} near a bubble as a function of time. The adsorbed PFOS, n_{PFOS}, linearly increases after 5 s. When C_{PFOS, bulk} was set as 10⁻¹¹ M, n_{PFOS} reaches 1.3×10⁻¹⁰ mol at t=20 s, accounting for 29% of the total available PFOS in the source. When a bubble bursts, it typically forms 5 aerosol droplets with sizes of 10% of the bubble size (Kientzler, et al., Tellus 1954, 6, 1-7). In other words, the ratio between the total volume of aerosol droplets and the corresponding bubble volume (Vₐₑᵣₒₛₒₗ/V_{bubble}) is 0.005. For a 70-μm-radius bubble, Vₐₑᵣₒₛₒₗ should be 7.2×10⁻¹² L, giving a final C_{PFOS, aerosol} of 1.8×10⁻⁸ M, and an R of 1800. This simulated R using the present disclosure's diffusion-limited model is close to the experimental value of 1140±90 at C_{PFOS, bulk}=10⁻¹¹ M in FIG. 17B.

Next, the analytical expression of enrichment rate (∂R/∂h) was derived. The simulation result in FIG. 27B shows that the diffusion of PFOS reaches a quasi-steady state after 5 s. This is caused by the spherical diffusion field of PFOS near a gas bubble. The PFOS depletion region's growth fails to significantly affect the concentration gradients at the surface because the diffusion field can draw material from a continually larger area at its outer limit (Bard, et al., “Electrochemical Methods: Fundamentals and Applications” Wiley: New York, 1980). The solution of the diffusion equation in a spherical diffusion field at steady state is given by (Bard, et al., “Electrochemical Methods: Fundamentals and Applications” Wiley: New York, 1980)

∂n_{PFOS}/∂t=(A_{bubble}D_{PFOS}C_{PFOS,bulk})/r_{bubble} Eq. 9:

where D_{PFOS} is the diffusion coefficient of PFOS in water, and A_{bubble} is the surface area of a bubble. The expression of C_{PFOS}, aerosol, was obtained after approximating t as the ratio of h to the average bubble velocity (vbubble) and using the empirical Vaerosol/Vbubble ratio 0.005 described above (Kientzler, et al., Tellus 1954, 6, 1-7).

C_{PFOS,aerosol}=(200A_{bubble}D_{PFOS}C_{PFOS,bulk}h)/r_{bubble}v_{bubble}V_{bubble} Eq. 10:

which can be then rearranged to yield

∂R/∂h=(200A_{bubble}D_{PFOS})/r_{bubble}v_{bubble}V_{bubble} Eq. 11:

As v_{bubble} can be estimated by Stokes' law (Batchelor, et al., Cambridge University Press, 1967), Eq. 11 is simplified as

∂R/∂h=2.4×10⁻⁴[m²·s](D_{PFOS})/r⁴_{bubble} Eq. 12:

Using D_{PFOS}=0.4×10⁻⁹ m²/s (Pereira, et al., Chem. Eng. Data 2014, 59, 3151-3159) and r_{bubble}=70 μm, Eq. 12 predicts an enrichment rate of 40 cm⁻¹, which is close to the experimental value of 30 cm⁻¹ in FIG. 25. The great agreement between the present disclosure's prediction and experimental data again confirms the present disclosure's diffusion-limited adsorption model. This model may underestimate the enrichment rate for bubbles generated by pushing high-pressure gas through a microporous membrane where substantial convective mass transfer, as well as the turbulent flow, can take place.

Next, the enrichment plateau at C_{PFOS, bulk}=10⁻⁸ M, and h>20 cm (FIG. 25) was analyzed. This plateau indicates the PFOS in the solution and on the bubble surface have reached an equilibrium. To further confirm this, the air-water interface adsorption coefficient, K, was estimated by:

K=(Γ_{PFOS})/C_{PFOS,bulk}≈R(0.005V_{bubble})/A_{bubble} Eq. 13:

where Γ_{PFOS} is the surface excess concentration of PFOS at equilibrium. A K value of 0.011 cm for a 70-μm-radius gas bubble was obtained. This value is in good agreement with the literature value of 0.014 cm (Chen, W., “Adsorption and retardation of PFAS in soils” University of Arizona, 2018; Lyu, et al., Environ. Sci. Technol. 2018, 52, 7745-7753), confirming the enrichment plateau is caused by the adsorption equilibrium of PFOS.

Another interesting finding is the large intercepts of the R vs. h plots in FIG. 25. Ata bubble path close to zero, the enrichment factor is still as high as 250, accounting for 25% of the total enrichment. In comparison, Chen and co-workers only observed an enrichment factor of 30 at zero-bubble path using their aerosol enrichment setup (Chingin, et al., ACS Omega 2018, 3, 8709-8717). This difference may be caused by electrode-PFOS interactions, which increase local PFOS concentration near the electrode surface.

Modulating the Preconcentration by Electrochemistry. According to Eq. 12, the enrichment rate (∂R/∂h) is inversely proportional to the fourth-order bubble radius (r⁴_{bubble}). To test this, bubble size was tuned by applying different voltages. FIG. 16 shows the average r_{bubble} increases slowly with the applied voltage from 0.045 mm at 30 V to 0.064 mm at 80 V. The corresponding current increased from 0.31 to 0.98 A, the enrichment factor, R, decreased from 2600 to 1200. The enrichment rate, ∂R/∂h, decreased from 100 to 30 cm-1 (FIG. 28). After fitting the plot of ∂R/∂h vs. r_{bubble} in the form of Eq. 12, a D_{PFOS} value of 0.22×10⁻⁹ was obtained, which is in reasonable agreement with the literature value of 0.4×10⁻⁹ m²/s (Pereira, et al., Chem. Eng. Data 2014, 59, 3151-3159). The good agreement confirms the present disclosure's model and also indicates that the enrichment process can be readily modulated by electrochemistry.

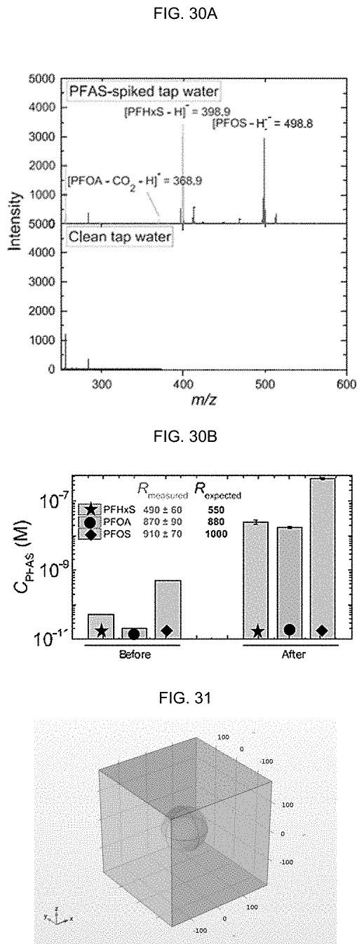

Preconcentration of PFAS in Tap Water. To test the performance of this preconcentration method for real-world samples, tap water was collected at Wayne State University's chemistry building. The tap water was spiked with a mixture of PFHxS, PFOA, and PFOS. The PFAS concentrations in the unspiked tap water were below the mass spectrometer's detection limit (FIG. 29). Even after preconcentration, no PFAS signal was observed (FIG. 30A). The spiked tap water contains 0.04 nM (16 ng/L) PFHxS, 0.02 nM (8 ng/L) PFOA, and 0.2 nM (100 ng/L) PFOS. This PFAS pattern is the same as that found in Robinson Elementary School's drinking water on Oct. 29, 2018, in Grand Haven, Mich. (The State of Michigan, Robinson Elementary School Drinking Water Response). After preconcentration, the mass spectrum showed the signals of PFHxS, PFOA, and PFOS (FIG. 30A). The C_{PFAS} in the aerosol sample was then calculated from these mass spectrometry signals, yielding an enrichment factor, R_{measured}, of 490±60, 870±90, and 910±70 for PFHxS, PFOA, and PFOS, respectively. These R_{measured} values are in good agreement (10% difference) with the R_{expected} values determined using the standard solutions (FIGS. 17B and 24). The consistent enrichment performance indicates the present disclosure's analytical utility's preconcentration method in analyzing ultralow concentration PFAS in real-world water samples.

In conclusion, a simple and efficient method for preconcentrating PFAS in water via electrochemical aerosol formation was presented. A 1000-fold enrichment of 10 common PFAS was demonstrated within 10 min. For each PFAS, the enrichment factor shows less than 10% variation at the concentration range of 10⁻¹² to 10⁻⁹ M. A diffusion-limited adsorption theory for this electrochemical aerosol enrichment method was built and validated. Using this method to preconcentrate PFAS in tap water was also demonstrated, indicating it is useful for addressing the current challenges in analyzing ultralow concentration PFAS in water.

Supporting Information. Diffusion-Limited Adsorption Model. Fick's laws of diffusion were used to simulate the change of C_{PFOS} as a function of time. In the three-dimension model, there was a solution cube with a side length of 360 μm and a 70-μm-radius gas bubble sphere at the cube center (FIG. 31). The initial concentration of PFOS in the cube was set as 10⁻¹¹ M. No mass flow in or out the cube walls. It was assumed the gas bubble served as a PFOS sink. When a PFOS molecule arrives at the gas bubble's surface, it gets absorbed immediately and does not leave. The equivalent C_{PFOS} at the bubble surface can be considered as 0, which was used as the concentration boundary condition. The diffusion coefficient of PFOS was set as 0.4×10⁻⁹ m²/s (Pereira, et al., J. Chem. Eng. Data 2014, 59 (10), 3151-3159). The statistical measurements of the partial mesh used in the finite element simulation were: mesh vertices (358265); tetrahedral elements (2099881); triangular elements (38308); edge elements (792); vertex elements (14). The domain element statistics were as followed: number of elements, 2099881; minimum element quality, 0.2087; average element quality, 0.6633; element volume ratio, 0.05233; and mesh volume, 4.522×10⁷ μm³.

(vi) Closing Paragraphs. Unless otherwise indicated, the practice of the present disclosure can employ conventional techniques of chemistry, organic chemistry, biochemistry, analytical chemistry, physical chemistry, and electrochemistry. These methods are described in the following publications. See, e.g., Harcourt, et al., Holt McDougal Modern Chemistry: Student Edition (2018); J. Karty, Organic Chemistry Principles and Mechanisms (2014); Nelson, et al., Lehninger Principles of Biochemistry 5th edition (2008); Skoog, et al., Fundamentals of Analytical Chemistry (8th Edition); Atkins, et al., Atkins' Physical Chemistry (11th Edition); Lefrou, et al., Electrochemistry: The Basics, with Examples, 2012.

The present disclosure provides computer control systems that are programmed to implement methods of the disclosure, including applications of voltage to electrodes, monitoring of current, observation of peak current value and drop, and/or correlation of observed values with reference levels within a database. As indicated, the computer system includes a processor (also central processing unit (CPU) or “computer processor” herein), which can be a single-core or multi-core processor or a plurality of processors for parallel processing. The computer system can also include memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface, and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network, in some cases, is a telecommunication and/or data network. The network can include one or more computer servers, which can enable distributed computing, such as cloud computing. The network, in some cases, with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries, and saved programs. The storage unit can store user data, e.g., user preferences and user programs. The computer system, in some cases, can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user. Examples of remote computer systems include personal computers (e.g., portable PC), slate or tablet PC's (e.g., Apple® iPad, Samsung® Galaxy Tab), telephones, Smartphones (e.g., Apple® iPhone, Android-enabled device, Blackberry®), or personal digital assistants. The user can access the computer system via the network.

Methods, as described herein, can be implemented by way of machine (e.g., computer processor) executable code stored on an electronic storage location of the computer system, such as, for example, on the memory or electronic storage unit. The machine-executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on the memory.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture,” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives, and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including a tangible storage medium, a carrier wave medium, or a physical transmission medium. Non-volatile storage media include, for example, optical or magnetic disks, such as any of the storage devices in any computer(s) or the like, such as may be used to implement the databases, etc., shown in the drawings. Volatile storage media include dynamic memory, such as the main memory of such a computer platform. Tangible transmission media include coaxial cables; copper wire, and fiber optics, including the wires that include a bus within a computer system. Carrier-wave transmission media may take the form of electric or electromagnetic signals, or acoustic or light waves such as those generated during radio frequency (RF) and infrared (IR) data communications. Common forms of computer-readable media, therefore, include, for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system can include or be in communication with an electronic display that includes a user interface (UI) for providing, for example, results of surfactant identification and concentration within an aqueous solution. Examples of UI's include a graphical user interface (GUI) and a web-based user interface.

Methods and systems of the present disclosure can be implemented by way of one or more algorithms. An algorithm can be implemented by way of software upon execution by the central processing unit. The algorithm can, for example, identify one or more surfactants and their respective concentrations within an aqueous solution as described herein.

As will be understood by one of ordinary skill in the art, each embodiment disclosed herein can comprise, consist essentially of or consist of its particular stated element, step, ingredient, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. A material effect would cause a statistically significant reduction in the ability to obtain a claimed effect according to a relevant experimental method described in the current disclosure. The present disclosure, for example, reports high specificity for detecting ultralow levels (ng/L) of surfactants in an aqueous solution, using bubble-nucleation preconcentration and mass spectrometry.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1-78. (canceled)

79. A method of assaying an aqueous solution for the presence of a surfactant and determining surfactant concentration if present, comprising:
obtaining an aqueous solution;
adding the aqueous solution to a container housing an electrode, wherein the electrode is submerged in the aqueous solution upon addition of the aqueous solution to the container;
applying a voltage to the electrode to produce gas bubbles; and
monitoring current values following application of the voltage, wherein at least one monitored current value correlates to the concentration of the surfactant within the aqueous solution;
thereby assaying the aqueous solution for the presence of the surfactant, and if present, determining the concentration of the surfactant within the aqueous solution based on the at least one monitored current value.

80. The method of claim 79, wherein the surfactant comprises perfluorooctanesulfonate, perfluorooctanoic acid, perfluorohexanoic acid, perfluoroheptanoic acid, perfluorooctanoate, perfluorononanoic acid, perfluorodecanoic acid, perfluoroundecanoic acid, perfluorododecanoic acid, perfluorobutane sulfonic acid, perfluorohexane sulfonic acid, perfluorooctane sulfonic acid, perfluoroalkyl substance, or polyfluoroalkyl substance.

81. The method of claim 79, wherein the aqueous solution comprises water, tap water, river water, seawater, lake water, glacier water, ocean water, salt water, natural water, sound water, strait water, channel water, gulf water, estuary water, polynya water, bay water, inlet water, shoal water, ice water, acid water, basic water, and/or rainwater.

82. The method of claim 79, wherein the aqueous solution comprises a buffer solution, an electrolyte, and/or a mixture of sodium perchlorate and perchloric acid.

83. The method of claim 79, wherein the container comprises at least a one compartment electrochemical cell.

84. The method of claim 79, wherein the electrode is a nanoelectrode having a radius between the range of 1 nm to 100 nm.

85. The method of claim 84, wherein the nanoelectrode:
is solid platinum, nickel, palladium, or gold;
comprises an anode and a cathode; and
has a radius between 5 nm to 100 nm.

86. The method of claim 79, comprising applying the voltage for 10 minutes or less with a generator.

87. The method of claim 79, comprising monitoring the current values with a digital reader at a constant scan rate.

88. The method of claim 79, wherein the at least one monitored current value comprises the peak current value and the minimal peak current value following the peak current value and the differential between the peak current value and the minimal peak current value following the peak current value, is inversely correlated to the concentration.

89. The method of claim 79, wherein the concentration of the surfactant is displayed on a user interface.

90. A method of preconcentrating a surfactant into a sample by forming and capturing an aerosol comprising:
applying a voltage to an electrode within an aqueous solution to generate gas bubbles that form an aerosol; and
capturing liquid from the formed aerosol thereby preconcentrating the surfactant into the sample.

91. The method of claim 90, wherein the preconcentrating provides a surfactant enrichment factor of 500 to 1300-fold over the concentration of the surfactant in the aqueous solution.

92. The method of claim 90, wherein the aqueous solution comprises water comprises tap water, river water, seawater, lake water, glacier water, ocean water, salt water, natural water, sound water, strait water, channel water, gulf water, estuary water, polynya water, bay water, inlet water, shoal water, ice water, acid water, basic water, and/or rainwater.

93. The method of claim 90, wherein the aqueous solution is within a container comprising at least one compartment electrochemical cell, a buffer solution, and an electrolyte.

94. The method of claim 90, wherein the electrode is solid platinum, nickel, palladium, or gold and comprises an anode and a cathode.

95. The method of claim 90, comprising applying the voltage with a generator.

96. The method of claim 90, comprising setting the depth of the electrode between 0 to 30 cm below the surface of the aqueous solution to create a selected bubble radius.

97. The method of claim 90, comprising capturing the aerosol via a platform positioned above the surface of the aqueous solution.

98. The method of claim 90, wherein the preconcentrated surfactant is analyzed by mass spectrometry.
